(19) United States
(12) Patent Application Publication
(10) Pub. No.: US 2023/0346589 A1
(43) Pub. Date: Nov. 2, 2023

(54) SYSTEMS AND METHODS FOR REMOVING BIOMATERIAL IMPLANTS

(71) Applicant: Contraline, Inc., Charlottesville, VA (US)

(72) Inventors: Gregory GROVER, Houston, TX (US); Kevin EISENFRATS, Charlottesville, VA (US); Tyler CHIARTAS, Charlottesville, VA (US); Robert GREENLEAF, Charlottesville, VA (US)

(73) Assignee: Contraline, Inc., Charlottesville, VA (US)

(21) Appl. No.: 17/923,115

(22) PCT Filed: May 27, 2021
(86) PCT No.: PCT/US2021/034562

§ 371 Date: Nov. 3, 2022

Related U.S. Application Data
(60) Provisional application No. 63/031,280, filed on May 28, 2020.

(51) Int. Cl. A61F 6/22; A61B 17/3207; A61L 31/14

(52) U.S. Cl. CPC A61F 6/22; A61B 17/320758; A61L 31/145; A61B 2217/007; A61B 2017/22094

(57) ABSTRACT

Methods for removing a biomaterial implant and reversing occlusive effects of the biomaterial implant are described. In particular, methods for performing removal of biomaterial implants without damage to a body lumen where the biomaterial was previously implanted are presented. More specifically, methods of reversing an implant within a vas deferens using an ablation device are described.

### CROSS-REFERENCE TO RELATED APPLICATIONS

This application claims benefit of priority to U.S. Provisional Application No. 63/031,280 entitled “Systems and Methods for Removing Biomaterial Implants,” filed May 28, 2020, which is incorporated herein by reference in its entirety.

This application is also related to U.S. patent application Ser. Nos. 16/681,572 and 16/681,577, each entitled “Systems and Methods for Delivering of Biomaterials” and each filed on Nov. 12, 2019, and to International Patent Application No. PCT/US2021/032235 entitled “Biomaterial Compositions and Methods of Delivery,” filed on May 13, 2021, each of which is incorporated herein by reference in its entirety.

### BACKGROUND

The embodiments described herein relate generally to methods and systems for removing biomaterials, and more particularly to removal of biomaterial implants from a body lumen.

Biomaterials are natural or synthetic materials (such as polymers) that are suitable for introduction into living tissues as therapeutics (to treat, augment, repair, modify, or replace a tissue function of the body) or as diagnostics. Biomaterials such as hydrogel implants have been shown to be useful for embolization, drug delivery, sealing, filling, and occlusion purposes. Hydrogels are highly hydrated polymer chains or networks that are able to absorb significant volumes of water and can have tunable mechanical properties. Biomaterials are often injectable, such as through a needle and/or catheter into the body. When injected, the material may gel or cross-link to form the implant.

Some known systems and methods include injecting and/or implanting a biomaterial product (e.g., a hydrogel) into a small area such as the lumen of a vessel or duct. For example, in some applications, the biomaterial will form an implant that acts as an occlusion or embolization of a lumen. The occlusion can be used for providing contraception to a subject and/or for reducing fertility and/or inducing infertility of the subject by occluding the vas deferens, fallopian tube(s), or uterus. Such occlusions can also be used to occlude any other body part, such as ducts, tissues, interstitial spaces, or organs such as for drug delivery, spacing, sealing, embolizing, or bulking purposes.

For contraceptive applications, there are currently no long acting, reversible contraceptives available for males on the market. For example, while vasectomy is long acting for contraceptive purposes, the procedure is generally considered permanent due to the difficulty in reversing the process. Even when the reversal procedure is correctly performed, patients often have low rates of fertility following reversal. By way of another example, a biomaterial may be implanted within the vas deferens to occlude the vas deferens. A surgical procedure can be performed where the vas deferens is cut along a location of the implant, the implant is removed, and the vas deferens is sutured back together. However, such a surgical removal procedure results in damage to the vas deferens and does not restore sperm parameters comparable to pre-implant baseline levels. In some known systems and methods where a biomaterial is implanted to occlude the vas deferens, the implant can be reversed by supplying a chemical solution to dissolve the implant within the vas deferens. However, such systems and methods are complicated to perform and also typically do not restore sperm parameters comparable to pre-implant baseline levels.

Thus, a need exists for systems and methods of removing biomaterial implants from a reproductive system to restore functionality and fertility of a patient to levels similar to those prior to the implantation of the biomaterial. A need further exists for a removal procedure that can be performed in a short time frame with consistent and repeatable results. A need further exists for systems and methods to be performed in a safe, minimally invasive manner to reduce recovery time of the patient following the removal procedure and to allow the patient to have multiple implants and reversals throughout their lifetime.

### SUMMARY

Systems and methods for removing a biomaterial implant and reversing occlusive effects of the biomaterial implant are described herein. In particular, methods for performing removal of biomaterial implants without damage to a body lumen where the biomaterial was previously placed are described.

In some embodiments, a method of removing an implant from a body lumen includes inserting a delivery member into the body lumen. The method includes conveying, via the delivery member, a distal tip of an ablation device into the body lumen. In some embodiments, the delivery member is an angiocatheter. The method further includes actuating the ablation device to rotate the distal tip within the body lumen. The method includes advancing the rotating distal tip within the body lumen to contact the implant to thereby ablate the implant. In some embodiments, the method includes conveying a flush solution into the body lumen. In some embodiments, the advancing of the rotating distal tip includes moving the distal tip through the implant in a first direction and then moving the distal tip through the implant in a second direction opposite the first direction. In some embodiments, the first direction is an upstream to downstream direction relative to the body lumen, and the second direction is a downstream to upstream direction relative to the body lumen. In some embodiments, the moving includes rotating the distal tip. In some embodiments, the moving includes oscillating the distal tip radially relative to a central axis of the distal tip. In some embodiments, the moving includes oscillating the distal tip axially relative to the central axis of the distal tip.

In some embodiments, the delivery member is inserted into the body lumen upstream from the implant. In some embodiments, the method includes advancing the rotating distal tip within the body lumen to contact the implant, thereby ablating the implant. In some embodiments, the method includes directing the flush solution to disburse the ablated implant within the body lumen. In some embodiments, the directing the flush solution advances the ablated implant in an upstream to downstream direction within the body lumen. In some embodiments, the directing the flush solution includes advancing the ablated implant to a portion of a urinary tract. In some embodiments, the method includes applying ultrasound waves to at least one of the distal tip, the body lumen, or the implant to detect a position of the distal tip relative to the implant.

In some embodiments, the method includes conveying a guide member and a driveshaft of the ablation device into the body lumen via the delivery member. The guide member includes an outer surface, an inner surface, and a guide lumen defined by the inner surface. The driveshaft extends through and is rotatably supported by the guide lumen. The distal tip of the ablation device is coupled to a distal end portion of the driveshaft. In some embodiments, the method further includes actuating a proximal end of the driveshaft to rotate in a direction about a centerline of the driveshaft. The driveshaft transfers a rotational force from the proximal end portion to the distal end portion to rotate the distal tip of the ablation device. In some embodiments, the direction of rotation is a first direction about the centerline of the driveshaft and the rotational force is a first rotational force. The method further includes actuating the proximal end portion of the driveshaft to rotate in a second direction about the centerline of the driveshaft, the second direction being opposite the first direction, and the driveshaft transfers a second rotational force from the proximal end portion to the distal end portion to reverse rotation of the distal tip portion of the ablation device.

In some embodiments, the method includes withdrawing the driveshaft and the distal tip of the ablation device from the body lumen through the guide member while the guide member is inserted into the delivery member. The method further includes conveying a flush solution into the body lumen through the guide lumen of the guide member. In some embodiments, the method includes inserting a supply tube into the body lumen through the delivery member and conveying a flush solution into the body lumen through the supply tube.

In some embodiments, a method of reversing an implant within the vas deferens that is occluding a flow of sperm through the vas deferens includes inserting a delivery member into the vas deferens. The method includes conveying, via the delivery member, a distal tip of an ablation device into the vas deferens. The method further includes actuating the ablation device to cause disruption, fragmentation, or removal of the implant from within the vas deferens. In some embodiments, the disruption, fragmentation, or removal of the implant with the ablation device results in at least one of A) a total sperm motility of sperm passing through the vas deferens after removal of the implant being substantially similar a total sperm motility before placing of the implant in the vas deferens, B) a total sperm concentration passing through the vas deferens after the disruption of the implant being substantially similar to a total sperm concentration before placing of the implant in the vas deferens, C) an ejaculate volume passing through the vas deferens after the disruption of the implant to be substantially similar to the ejaculate volume before placing of the implant in the vas deferens, or D) a forward progression of sperm through the vas deferens after the disruption of the implant being substantially similar to a forward progression of sperm before placing of the implant in the vas deferens. In some embodiments, the disruption, fragmentation, or removal of the implant with the ablation device results in at least one of: A) a total sperm motility of sperm passing through the vas deferens at the implant location after the disruption of the implant being at least 30% to 70% of a total sperm motility of the sperm passing through the vas deferens at a location upstream from the implant location, B) a total sperm concentration passing through the vas deferens at the implant location after the disruption of the implant being at least 30% to 70% of a total sperm concentration passing through the vas deferens at the location upstream from the implant location, or C) an ejaculate volume passing through the vas deferens at the implant location after the disruption of the implant being at least 30% to 70% of an ejaculate volume before passing through the vas deferens at the location upstream from the implant location. In some embodiments, the disruption, fragmentation, or removal of the implant with the ablation device results in at least one of: A) a total sperm motility of sperm passing through the vas deferens at the implant location after the disruption of the implant being at least 30% to 70% of a total sperm motility of the sperm passing through the vas deferens at a location upstream from the implant location, B) a total sperm concentration passing through the vas deferens at the implant location after the disruption of the implant being at least 30% to 70% of a total sperm concentration passing through the vas deferens at the location upstream from the implant location, or C) an ejaculate volume passing through the vas deferens at the implant location after the disruption of the implant being at least 30% to 70% of an ejaculate volume before passing through the vas deferens at the location upstream from the implant location. In some embodiments, the disruption, fragmentation, or removal of the implant with the ablation device results in at least one of A) a total sperm motility of sperm passing through the vas deferens at the implant location after the disruption of the implant being substantially similar to a total sperm motility of the sperm passing through the vas deferens at a location upstream from the implant location, B) a total sperm concentration passing through the vas deferens at the implant location after the disruption of the implant being substantially similar to a total sperm concentration passing through the vas deferens at the location upstream from the implant location, C) an ejaculate volume passing through the vas deferens at the implant location after the disruption of the implant to be substantially similar to an ejaculate volume before passing through the vas deferens at the location upstream from the implant location, or D) a forward progression of sperm through the vas deferens at the implant location after the disruption of the implant being substantially similar to a forward progression of sperm passing through the vas deferens at the location upstream from the implant location. In some embodiments, the disruption, fragmentation, or removal of the implant with the ablation device results in an effective diameter of an inner lumen of the vas deferens at the implant location after the disruption of the implant being substantially similar to the effective diameter of an inner lumen of the vas deferens at a location directly upstream from the implant location. In some embodiments, the disruption, fragmentation, or removal of the implant with the ablation device results in a total sperm motility of sperm passing through the vas deferens at the implant location after the disruption of the implant being at least about 25 micrometers per second. In some embodiments, the disruption, fragmentation, or removal of the implant with the ablation device results in an effective diameter of an inner lumen of the vas deferens at the implant location after the disruption of the implant being substantially similar to the effective diameter of an inner lumen of the vas deferens at a location directly upstream from the implant location. In some embodiments, the disruption, fragmentation, or removal of the implant with the ablation device results in restoration of flow through the vas deferens at the implant location. In some embodiments, the disruption, fragmentation, or removal of the implant with the ablation device enables a flush solution to be conveyed through the implant location of the vas deferens, after the disruption of the implant, with a force of less than or equal to about 44.48 N (10 lbF). In some embodiments, the disruption, fragmentation, or removal of the implant with the ablation device enables at least about 0.1 mL to about 10 mL of flush solution to be conveyed into and through the vas deferens after the disruption of the implant.

In some embodiments, the total sperm motility of sperm passing through the vas deferens after removal of the implant is at least about 30% to 70% (e.g., not more than a about 30-70% reduction or difference) the total sperm motility before placing of the implant in the vas deferens. In some embodiments, the total sperm motility of sperm passing through the vas deferens after removal of the implant is at least about 60% to 70% (e.g., not more than about 30-40% reduction or difference) the total sperm motility before placing of the implant in the vas deferens. In some embodiments, the total sperm motility of sperm passing through the vas deferens after removal of the implant is at least about 70% to 80% (e.g., not more than about 20-30% reduction or difference) the total sperm motility before placing of the implant in the vas deferens. In some embodiments, the total sperm concentration passing through the vas deferens after removal of the implant is at least about 85% to 95% (e.g., not more than about 5-15% reduction or difference) the total sperm concentration before placing of the implant in the vas deferens. In some embodiments, the removal of the implant with the ablation device results in a post-implant motility of sperm passing through the vas deferens after removal of the implant being sufficient to travel through a female reproductive tract and to fertilize an egg. In some embodiments, proteins and organelle (e.g., acrosome) of sperm passing through the vas deferens after the removal of the implant remain unaltered in substantially the same fashion as proteins and organelle of sperm passing through the vas deferens prior to the implant.

In some embodiments, the method includes actuating the ablation device to rotate the distal tip while contacting a portion of the implant. The rotational contact between the distal tip and the portion of the implant results in fragments from the portion of the implant being mechanically separated from the implant. In some embodiments, the method includes actuating the ablation device in a first rotational direction while in contact with the implant, and actuating the ablation device in a second rotational direction, opposite of the first rotational direction, while in contact with the implant. In some embodiments, the method includes conveying a flush solution into the vas deferens, via the delivery member, to advance the ablated implant out of the vas deferens. In some embodiments, the method includes applying ultrasound waves to at least one of the distal tip, the body lumen, or the implant to detect a position of the distal tip relative to the implant.

In some embodiments, the method includes advancing the distal tip of the ablation device through at least a central portion of the implant. The method further includes displacing the central portion of the implant to cause at least a portion of an outer perimeter of the implant contacting the vas deferens to contract and collapse towards a center of the vas deferens. The collapse of the implant reduces the effective outer diameter of the implant such that the reduced effective diameter is less than the effective inner diameter of the vas deferens. In some embodiments, the method further includes advancing the collapsed implant through the vas deferens and toward the urinary tract.

In some embodiments, the method includes reversing the implant in a manner which minimizes damage to an inner lining of the vas deferens (e.g., epithelial lining). The method can result in denuding of columnar nature of the lining, which is capable of regeneration. In some embodiments, the method prevents puncturing, tearing, or shredding of the adjacent muscle layers.

In some embodiments, an ablation device includes a distal tip. Use of the ablation device includes removing an implant from a lumen. The distal tip is configured to be conveyed into the lumen via a delivery member that has been inserted into the lumen. The distal tip of the ablation device is configured to be: A) rotated when the ablation device is actuated and B) advanced into the lumen to ablate the implant within the lumen. A flush solution is configured to be conveyed into the lumen. In some embodiments, the flush solution is a saline solution. In some embodiments, the flush solution is a phosphate buffered saline solution.

In some embodiments, the rotating distal tip of the ablation device is configured to be advanced through an entire length of the implant. In some embodiments, the rotating distal tip of the ablation device is configured to be withdrawn through the entire length of the implant. In some embodiments, the rotating distal tip of the ablation device is configured to be advanced through the entire length of the implant a second time after being withdrawn through the entire length of the implant. In some embodiments, the rotating distal tip of the ablation device is configured to be advanced through a central portion of the implant. The rotating distal tip of the ablation device is configured to displace the central portion of the implant to cause at least a portion of an outer perimeter of the implant to contract and collapse towards a center of the lumen.

In some embodiments, the delivery member defines a conduit through which the flush solution is conveyed. The delivery member is configured to direct the flush solution to advance the ablated implant in an upstream to downstream direction relative to a direction of flow within the lumen. In some embodiments, the delivery member defines a conduit and an outlet through which the flush solution is conveyed. The delivery member is configured to direct the flush solution out of the outlet to advance the ablated implant through the lumen and away from the outlet. In some embodiments, the implant includes a viscoelastic material.

In some embodiments, the implant is a hydrogel. In some embodiments, the hydrogel is a cross-linked hydrogel formed from a first component and a second component. In some embodiments, the first component is at least one of a polyvinyl alcohol, alginate or modified alginate, chitosan or modified chitosan, polyethyleneimine, carboxymethyl cellulose, or polyethylene glycol terminated with a biorthogonal functional group, and the second component is at least one of a water or buffer, water or buffer with divalent cations such as calcium, a solution of reduced hyaluronic acid, a solution of polystyrene sulfonate, a solution of gelatin, and/or polyethylene glycol terminated with a biorthogonal functional group.

In some embodiments, the lumen is defined by an inner surface of a tubing material. In some embodiments, the tubing material includes one or more of polyethylene (PE) tubing, polytetrafluoroethylene (PTFE) tubing, polydimethylsiloxane (PDMS) tubing, and/or silicon tubing. In some embodiments, the inner surface of the tubing material has a diameter of about 0.8 mm.

In some embodiments, an ablation device comprises a rotatable distal tip for use in treatment of a blockage within a lumen. The distal tip is configured to be conveyed into the lumen via a delivery member that has been inserted into the lumen. The distal tip of the ablation device is configured to be: A) rotated when the ablation device is actuated and B) advanced into the lumen to ablate the blockage within the lumen. A flush solution is configured to be conveyed into the body lumen to advance the ablated blockage in an upstream to downstream direction relative to a direction of flow within the lumen.

In some embodiments, the blockage is an implant. In some embodiments, the implant includes a hydrogel. In some embodiments, the hydrogel is a cross-linked hydrogel formed from a first component and a second component. In some embodiments, the first component is at least one of a polyvinyl alcohol, alginate or modified alginate, chitosan or modified chitosan, polyethyleneimine, carboxymethyl cellulose, or polyethylene glycol terminated with a biorthogonal functional group, and the second component is at least one of a water or buffer, water or buffer with divalent cations such as calcium, a solution of reduced hyaluronic acid, a solution of polystyrene sulfonate, a solution of gelatin, and/or polyethylene glycol terminated with a biorthogonal functional group.

In some embodiments, the distal tip includes one or more of an abrasive tip, a cutting tip, a milling tip, a grinding tip, a coring tip, or a boring tip. In some embodiments, the distal tip includes one or more a diamond, gold, aluminum, steel, titanium nitride, tungsten carbide, boron carbide, or silica material.

In some embodiments, the lumen is a body lumen. In some embodiments, the body lumen is a vas deferens, and the blockage is an implant within the vas deferens. In some embodiments, the lumen is defined by an inner surface of a tubing material. In some embodiments, the tubing material includes one or more of polyethylene (PE) tubing, polytetrafluoroethylene (PTFE) tubing, polydimethylsiloxane (PDMS) tubing, and/or silicon tubing. In some embodiments, the inner surface of the tubing material has a diameter of about 0.8 mm.

The description below and the accompanying figures will provide greater details on the various systems, methods for removing biomaterial implants.

### BRIEF DESCRIPTION OF THE DRAWINGS

FIG. 1 is a schematic illustration of a delivery member inserted into a body lumen occluded by a biomaterial implant according to an embodiment.

FIG. 2 is a schematic illustration of a distal tip of an ablation device inserted into the body via the delivery member of FIG. 1.

FIG. 3 is a schematic illustration of an enlarged view of the body lumen showing the distal tip of the ablation device in FIG. 2 being actuated to ablate the biomaterial implant.

FIG. 4 is a schematic illustration of an enlarged view of a body lumen showing a flushing fluid being directed towards an ablated biomaterial implant according to an embodiment.

FIG. 5 is a flow chart of a method of reversing or otherwise disrupting a biomaterial implant from a body lumen according to an embodiment.

FIG. 6 is a flow chart of a method of reversing or otherwise disrupting an implant within a vas according to an embodiment.

FIG. 7 is an illustration of a perspective view of a test arrangement showing an ablation device inserted into a simulated body lumen via a delivery member according to an embodiment.

FIG. 8 is an illustration of an enlarged side view of FIG. 7 showing the ablation device inserted through the delivery member.

FIG. 9 is an illustration of an enlarged side view of FIG. 7 showing a distal tip of the ablation device within the simulated body lumen.

FIG. 10 is an illustration of an enlarged cross-sectional view of FIG. 7 taken at A-A showing an interior surface of the simulated body lumen after removal of an ablated biomaterial implant.

FIGS. 11A and 11B are block graphs showing sperm count results of test subjects prior to and after removal of biomaterial implants.

FIGS. 12A and 12B are block graphs showing sperm motility results of test subjects prior to and after removal of biomaterial implants.

FIGS. 13A and 13B are block graphs showing ejaculate volume results of test subjects prior to and after removal of biomaterial implants.

FIG. 14A is an illustration of a canine vas deferens after an implant reversal procedure was performed.

FIG. 14B is an illustration of the canine vas deferens shown in FIG. 14A with blue-dye solution supplied through the vas deferens after the implant reversal.

### DETAILED DESCRIPTION

As generally described in related U.S. patent application Ser. Nos. 16/681,572 and 16/681,577, each entitled “Systems and Methods for Delivering of Biomaterials,” and in related International Patent Application No. PCT/US2021/032235 entitled “Biomaterial Compositions and Methods of Delivery, a first component is formulated to crosslink with a second component to form a biomaterial that can be implanted into or onto a body lumen, or other cavity, space, tissue or organ of a body. A delivery apparatus is used to inject the first and the second components such that the first and second components are mixed and cross-linked to form the biomaterial. The biomaterial may be formed and extruded into a body lumen or formed directly in the body lumen. The biomaterial may continue to gel and/or cross-link in situ once injected or can be completely gelled or cross-linked by the time it exist the apparatus. In this regard, the delivery apparatus facilitates the merging or mixing of the two or more different solutions into a single stream. In some embodiments, the selected body lumen is a vas deferens, and the biomaterial is delivered and implanted in the vas deferens to occlude the vas deferens and serve as contraception. In some embodiments, the selected body lumen is a fallopian tube, and the biomaterial is delivered and implanted in the fallopian tube to occlude the vas deferens and serve as contraception.

In some embodiments, the first component and the second component are each water soluble components. In some embodiments, the first component and the second component are capable of crosslinking to form the hydrogel. In some embodiments, the hydrogel formed by crosslinking the first component and the second component is at least 90 percent water. In some embodiments, the first component is characterized by having a first viscosity. The second component is characterized by a second viscosity, and the second viscosity is within 25 percent of the first viscosity. In some embodiments, the hydrogel formed by crosslinking the first component and the second component has a gelation time of less than 5 minutes.

In some embodiments, the conveying of the hydrogel out of the delivery apparatus includes conveying the hydrogel into or onto a body part, cavity, or lumen to at least partially occlude the body part or lumen. In some embodiments, the body part, cavity or lumen is one of an artery, vein, capillary, vessel, tissue, intra-organ space, lymphatic vessel, vas deferens, epididymis, fallopian tube, duct, bile duct, hepatic duct, cystic duct, pancreatic duct, parotid duct, organ, uterus, prostate, organ of a gastrointestinal tract or circulatory system or respiratory system or nervous system, subcutaneous space, intramuscular space, or interstitial space. In some embodiments, the hydrogel conveyed to the body lumen at least partially occludes the body lumen. In some embodiments, the hydrogel can additionally or alternatively provide contraceptive effect to a subject or induce azoospermia or infertility in a subject. In some embodiments, the conveying the hydrogel out of the delivery apparatus is performed in less than 30 seconds. In some embodiments, the conveying the hydrogel out of the delivery apparatus includes conveying between about 50 microliters to about 2 milliliters to a lumen, cavity, space, tissue or organ of a body. In some embodiments, the conveying the hydrogel out of the delivery apparatus includes conveying between about 50 microliters and about 200 microliters to a lumen, cavity, space, tissue, or organ of a body in between about 5 seconds and about 20 seconds. Thus, the methods of implant removal described herein can be performed to remove an implant from any of these body lumens.

Any of the methods of removal described herein can be performed to remove any of the biomaterials described herein. For example, any of the methods can be used to remove (or reverse implantation of) any of the hydrogels described herein. In some embodiments, the hydrogel is echogenic and the method includes identifying the bolus of air via an image of the body lumen BL, such as by ultrasound. In some embodiments, the body lumen is one of an artery, vein, capillary, vessel, tissue, intra-organ space, lymphatic vessel, vas deferens, epididymis, fallopian tube, duct, bile duct, hepatic duct, cystic duct, pancreatic duct, parotid duct, organ, uterus, prostate, organ of a gastrointestinal tract or circulatory system or respiratory system or nervous system, subcutaneous space, intramuscular space, or interstitial space.

In some embodiments, the hydrogel is conveyed out of the exit opening of the delivery apparatus into a body lumen to at least partially occlude the body lumen. In some embodiments, the body lumen is one of an organ of a reproductive system. In some embodiments, the body lumen is one of a vas deferens or a fallopian tube. In some embodiments, the body lumen has an inner diameter of about 5.0 mm or less. In some embodiments, the body lumen is surrounded by smooth muscles.

In some embodiments, the first component and the second component can be any of the biomaterial components described herein. For example, in some embodiments, the first component and the second component can each be a water soluble component (e.g., monomer, macromer, polymer, or the like) that is capable of crosslinking (e.g., with the other component) to form a hydrogel (as the delivered biomaterial product). In some embodiments, the first component and the second component are formulated such that the resulting hydrogel has a gelation time of less than 5 minutes. In other embodiments, the first component and the second component are formulated such that the resulting hydrogel has a gelation time of less than 2 minutes. In yet other embodiments, the first component and the second component are formulated such that the resulting hydrogel has a gelation time of less than 30 seconds. In some embodiments, the first component is at least one of a polyvinyl alcohol, alginate or modified alginate, chitosan or modified chitosan, polyethyleneimine, carboxymethyl cellulose, and/or polyethylene glycol terminated with one or more biorthogonal functional group (e.g., amine, thiol, maleimide, azide, activated ester). The second component is at least one of a water or buffer, water or buffer with divalent cations such as calcium, a solution of reduced hyaluronic acid, a solution of polystyrene sulfonate, a solution of gelatin, and/or polyethylene glycol terminated with one or more biorthogonal functional group (e.g., amine, thiol, maleimide, azide, activated ester). In some embodiments, polyvinyl alcohol, alginate, chitosan, polyethyleneimine, carboxymethyl cellulose, polyethylene glycol terminated with functional groups, divalent cations, reduced hyaluronic acid, polystyrene sulfonate, or gelatin have a weight percent ranging from about 1 to 30% in solvent, such as about 2 to 10%, about 3 to 12%, about 4 to 15%, about 5 to 20%, about 6 to 25%, or about 7 to 28%, or any range in between any of these endpoints. In some embodiments, the polysaccharides may be modified with one or more functional groups, such as the same or different functional groups. For example, the functional groups may include one or more of alcohols, amines, thiols, carboxylic acids, carboxylic acid derivatives, carbonates, carbamates, carbamides, alkanes (n=2 to n=12), alkenes, alkynes, maleimides, sulfones, vinyl sulfones, and activated carboxylic acids. In some embodiments, the polysaccharides and proteins may range in molecular weight from about 10,000 to about 1,000,000 grams/mole, such as about 15,000 to about 900,000 grams/mole, about 20,000 to about 850,000, about 25,000 to about 800,000, about 30,000 to about 700,000, about 50,000 to about 600,000, about 75,000 to about 500,000, about 100,000 to about 400,000, about 200,000 to about 300,000, or about 225,000 to about 275,000. In some embodiments, the polyvinyl alcohol, polystyrene sulfonate, polyethyleneimine, and polyethylene glycol may be linear, Y-shaped, 3-arm, 4-arm, 6-arm, or 8-arm and range in molecular weight from about 1,000 to about 1,000,000 grams/mole such as about 1,500 to about 900,000 grams/mole, about 2,000 to about 850,000, about 2,500 to about 800,000, about 3,000 to about 700,000, about 5,000 to about 600,000, about 7,500 to about 500,000, about 10,000 to about 450,000, about 15,000 to about 50,000, about 100,000 to about 400,000, about 200,000 to about 300,000, or about 225,000 to about 275,000 grams/mole, or any range in between any of these endpoints. The hydrogel can be any of the hydrogels described herein and can have any of the characteristics as indicated herein. For example, in some embodiments, the formed hydrogel can be at least 90 percent water, such as 92%, 93%, 94%, 95%, 96%, 97%, 98%, or 99%, or any range in between. In other embodiments, the formed hydrogel can be >50% water.

In some embodiments, the dissolving solution for the polymer component(s) may be aqueous buffers, including any one or more of phosphate, citrate, acetate, histidine, lactate, tromethamine, gluconate, aspartate, glutamate, tartrate, succinate, malic acid, fumaric acid, alpha-ketoglutaric, and/or carbonate. Specific solvents/buffers can include: 1) acetic acid and sodium acetate (AA), 2) citric acid and sodium citrate (CP), 3) citric acid and phosphate buffer (CP), and 4) phosphate buffer (PB). Non-aqueous solvents include: dimethyl isosorbide, glycofurol 75, PEG 200, diglyme, tetrahydrofurfuryl alcohol, ethanol, acetone, solketal, glycerol formal, dimethyl sulfoxide, propylene glycol, ethyl lactate, N-methyl-2-pyrrolidone, dimethylacetamide, methanol, isopropanol, 1,4-butanediol, ethyl acetate, toluene, acetonitrile. The molarity of the solutions/solvents/buffers can range for example from about 0.1 M to about 0.15 M to about 0.2 M, such as about 0.12 M to about 0.17 M to about 0.19 M, or any range in between any of these endpoints. In some embodiments, the solution can include about a 0.2 M citric acid buffer and can be formulated to have a solution pH of between 4.0 and 6.0. In some embodiments, the pH of the solution can be between 4.0 and 5.25. In some embodiments, the pH of the solution can be about 4.0. In other embodiments, the pH of the solution can be about 5.25. In yet other embodiments, the pH of the solution can be between about 4.5 and about 8 such as a pH of about 5-7, or about 4.5-6.

The term “about” when used in connection with a referenced numeric indication means the referenced numeric indication plus or minus up to 10% of that referenced numeric indication. For example, “about 100” means from 90 to 110.

The term “substantially” when used in connection with, for example, a geometric relationship, a numerical value, amount, and/or a range, such as with respect to a concentration, a volume, and/or movement/rate/speed, is intended to convey that the geometric relationship (or the structures described thereby), the number, and/or the range so defined is nominally the recited geometric relationship, number, and/or range. For example, two structures described herein as being “substantially parallel” is intended to convey that, although a parallel geometric relationship is desirable, some non-parallelism can occur in a “substantially parallel” arrangement. By way of another example, a structure defining a volume that is “substantially 0.50 milliliters (mL)” is intended to convey that, while the recited volume is desirable, some tolerances can occur when the volume is “substantially” the recited volume (e.g., 0.50 mL). Such tolerances can result from manufacturing tolerances, measurement tolerances, and/or other practical considerations (such as, for example, minute imperfections, age of a structure so defined, a pressure or a force exerted within a system, and/or the like). As described above, a suitable tolerance can be, for example, of ±10% of the stated geometric construction, numerical value, and/or range.

As used herein, the term “biomaterial component” (also referred to as “component”) includes any substance that is used in connection with any of the systems or delivery devices described herein to form a delivered biomaterial product. For example, a component can include a small molecule, catalyst, peptide, protein, enzyme, nucleotide (or derivatives of), short chains of nucleotides (or derivatives of), long chains of nucleotides (or derivatives of), monosaccharides (or derivatives of), disaccharides (or derivatives of), trisaccharides (or derivatives of), oligo saccharides (or derivatives of), polysaccharides (or derivatives of), monomer, oligomer, macromer, or polymer that can be cross-linked with another component to form a delivered product (e.g., hydrogel). A component can include a mixture or solution of one or more constituents (e.g., a polymer and a solvent). A component can include such constituents regardless of their state of matter (e.g., solid, liquid or gas). A component can include both active constituents and inert constituents. For example, in some embodiments, a component can include certain polymers that can form a delivered product, as well as a medicament or other active ingredient. By way of another example, in some embodiments, a component can include drugs, including but not limited to, small molecule drugs and biologics. In other embodiments, a component can include certain constituents to impart desired properties to the delivered product, including constituents that facilitate the delivered product being echogenic, radiopaque, radiolucent, or the like.

The term “biomaterial product,” “delivered biomaterial product,” or “delivered product” includes any substance that is delivered by any of the systems or delivery devices described herein. For example, a delivered product can be a biomaterial that is formed from multiple biomaterial components and delivered with any of the delivery systems described herein and then delivered to target locations. Thus, a delivered product can be the implant or structure that is formed with the system by multiple biomaterial components that react together or assemble into higher order structures via covalent and/or non-covalent bonds or interactions, and that is delivered by the system. In certain situations, the biomaterial can be delivered by the system in a fully formed state to a target location. Although a delivered product can be considered fully formed (i.e., the chemical reactions between the biomaterial components are completed), it can still undergo certain changes (e.g., in vivo changes) after delivery. For example, a delivered biomaterial product can continue to absorb water and/or swell and/or can expel impurities. In some embodiments, a delivered biomaterial product can be a hydrogel that is formed by crosslinking of two or more biomaterial components. The term “hydrogel” can refer to any water-swollen (majority, >50%, of material mass is water), and cross-linked polymeric network produced by the reaction of one or more components (e.g., polymers, monomers) and/or a polymeric material that exhibits the ability to swell and retain a significant fraction of water within its structure, but will not dissolve in water.

As used herein, the term “set” can refer to multiple features or a singular feature with multiple parts. For example, when referring to set of walls, the set of walls can be considered as one wall with multiple portions, or the set of walls can be considered as multiple, distinct walls. Thus, a monolithically-constructed item can include a set of walls. Such a set of walls can include, for example, multiple portions that are either continuous or discontinuous from each other. A set of walls can also be fabricated from multiple items that are produced separately and are later joined together (e.g., via a weld, an adhesive, or any suitable method).

The term “gelation” refers to the transition of the hydrogel components from a soluble polymer of finite branches to a substance with infinitely large molecules. Similarly stated, “gelation” refers to the condition where the gel forms and after the components are combined. Thus, the gelation time refers to the time that it takes for the resulting hydrogel to substantially reach equilibrium.

The term “downstream” refers to the direction of an intended or normal flow of fluid within a body lumen or channel. The term “upstream” refers to the direction opposite of the downstream direction, or opposite the direction of the intended or normal flow of fluid within a body lumen or channel. For example, the downstream direction within the vas deferens is the direction from the testes towards the penile urethra.

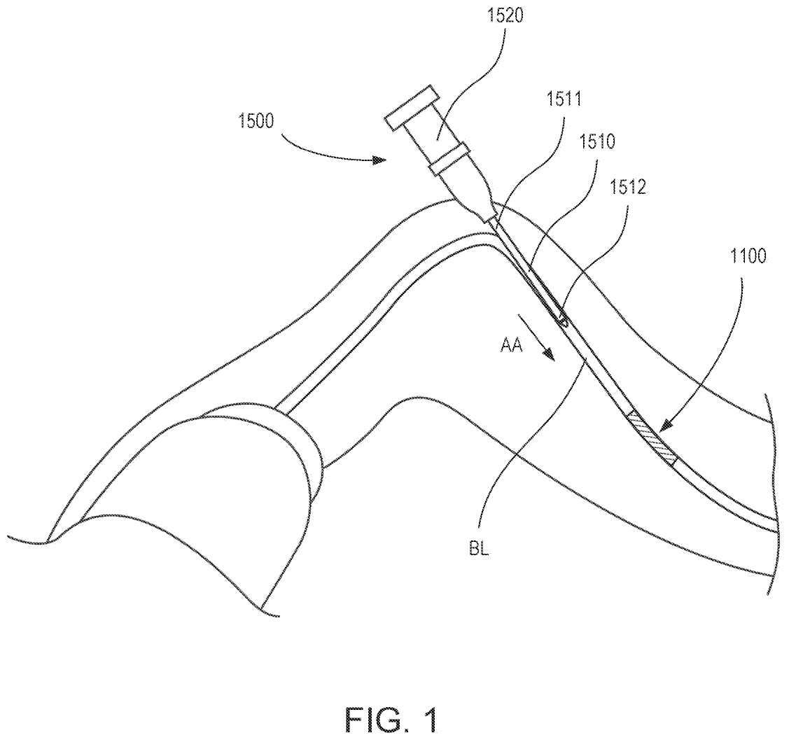

FIG. 1 shows a schematic illustration of a delivery member 1500 being inserted into a body lumen BL including an implant 1100 that occludes the body lumen BL. The delivery member 1500 includes a catheter 1510 and a hub portion 1520. The catheter 1510 includes a proximal end portion 1511 and a distal end portion 1512. The proximal end portion 1511 is coupled to the hub portion 1520. The distal end portion 1512 of the catheter 1510 is inserted into a body lumen BL and the distal end portion 1512 extends towards a downstream direction of the body lumen BL, as shown by the arrow AA. In some embodiments, the distal end portion 1512 is inserted into the body lumen BL upstream of the implant 1100 such that fragments of the implant 1100 can be conveyed downstream towards a natural outlet, such as a urinary tract, of a patient as will be described in greater detail below. In some embodiments, the delivery member 1500 is an angiocatheter.

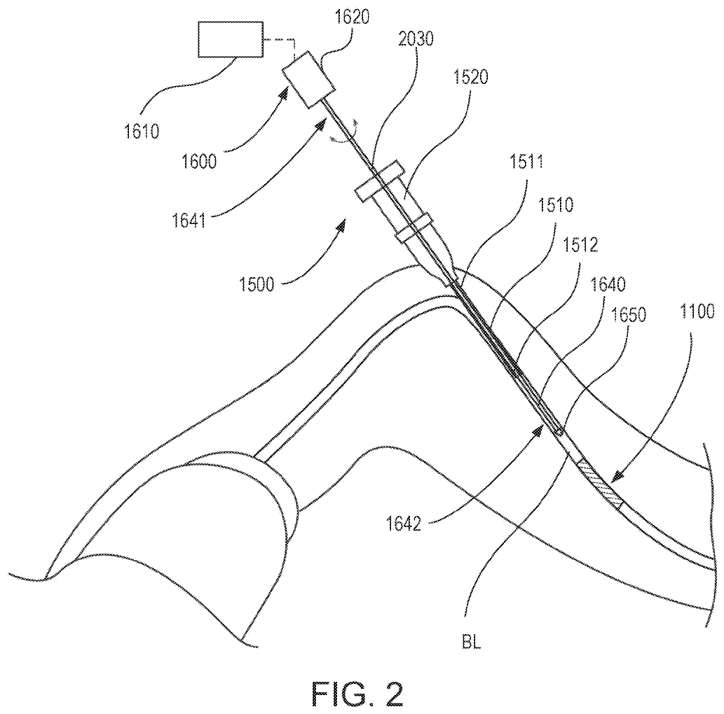

As shown in FIG. 2, a removal tool 1600, such as an ablation device, is inserted into the body lumen BL via the delivery member 1500. The removal tool 1600 includes a control unit 1610, a motor 1620, a driveshaft 1640, and a tool member 1650. The control unit 1610 is operatively connected to the motor 1620 to control one or more of a rotational speed, rotational torque, or rotational direction of the motor 1620. In some embodiments, the control unit 1610 receives inputs from an operator to start or stop the motor 1620, to control a rotational direction of the motor 1620, and/or to control the speed of the motor 1620. The driveshaft 1640 includes a proximal end portion 1641 that is coupled to and driven by the motor 1620. The driveshaft 1640 includes a distal end portion 1642 that is coupled to and drives the tool member 1650.

In some embodiments, the removal tool 1600 can include an outer sleeve or guide member (not shown), which further includes a proximal end portion, a distal end portion, an outer surface, an inner surface, and a guide lumen defined by the inner surface. The distal end portion is configured to be inserted into the body lumen BL via the delivery member 1500. The guide lumen is configured to rotatably support the driveshaft 1640 and enable the driveshaft 1640 to longitudinally translate relative to the inner surface. For example, the guide lumen is configured to permit the distal end portion 1642 of the driveshaft 1640 to extend beyond the distal end portion of the guide member and permit the distal end portion 1642 of the driveshaft 1640 to be withdrawn into the distal end portion of the guide member. In some embodiments, the guide member is inserted into the body lumen BL, via the delivery member 1500, prior to the tool member 1650 and the driveshaft 1640 being inserted into the body lumen BL via the guide lumen of the guide member. In some embodiments, the guide member, the driveshaft 1640 and the tool member 1650 are inserted into the body lumen BL together, via the delivery member 1500, while the driveshaft 1640 is within the guide lumen of the guide member.

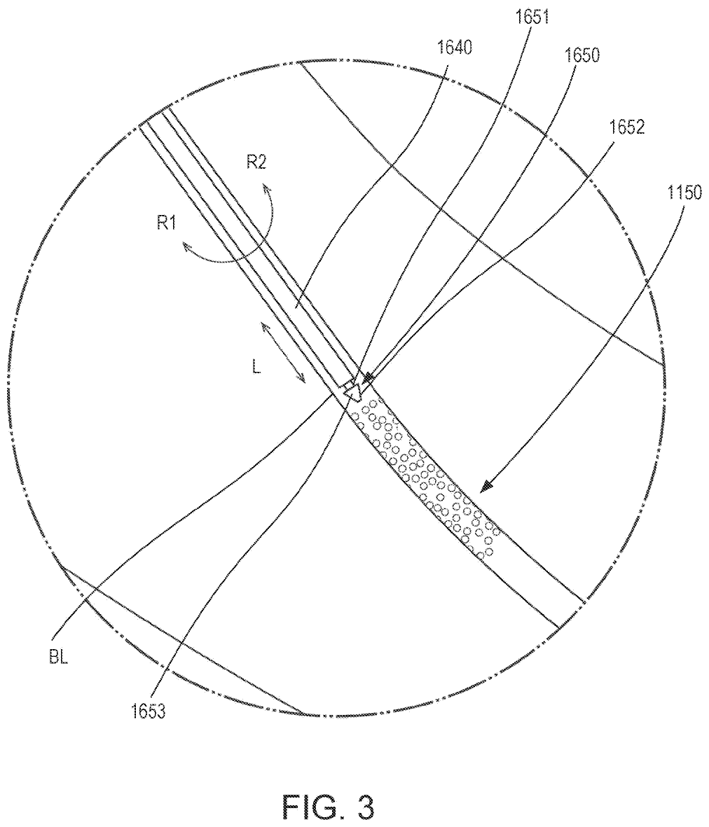

As shown in FIG. 3, the tool member 1650 includes a proximal driven portion 1651 and a distal tip portion 1652. The distal tip portion 1652 includes a working surface 1653 configured to ablate, cut, mill, and/or grind a material the working surface 1653 comes into contact with. In some embodiments, the distal tip portion 1652 is an abrasive tip, a cutting tip, a milling tip, a grinding tip, a coring tip, and/or a boring tip. In some embodiments, the distal tip portion 1652 is diamond coated tip. In some embodiments, the distal tip portion 1652 includes, but is not limited to, one or more of diamond, gold, aluminum, steel, titanium nitride, tungsten carbide, boron carbide, or silica material.

As shown in FIG. 3, the driveshaft 1640 is configured to orient and position the tool member 1650 within the body lumen BL near the implant 1100. The driveshaft 1640 is configured to transfer rotational force from the motor 1620 to the tool member 1650 and rotate the working surface 1653 in one or more of a first rotational direction R1 or a second rotational direction R2. The driveshaft 1640 is further configured to translate longitudinally L relative to a centerline of the driveshaft 1640 to advance the tool member 1650 against, into and/or through the implant 1100. As the tool member 1650 is advanced while rotating, the working surface 1653 contacts and ablates the implant 1100, causing portions or fragments 1150 of the implant 1100 to mechanically separate. Although the tool member 1650 is described as a mechanical ablation device, any of the tool members described herein can be a thermal and/or wave energy ablation device. In some embodiments, the tool member 1650 traverses an entire length of the implant 1100 to create a tunnel (not shown) such that an outer surface of the implant 1100 collapses inwards towards the tunnel and an effective outer diameter of the implant is smaller than an inner diameter of the body lumen BL. In some embodiments, the tool member 1650 traverses at least a portion of the entire length of the implant 1100 to create fragments that can easily be passed through the inner diameter of the body lumen BL. In some embodiments, the tool member 1650 traverses the entire length of the implant 1100 multiple times. Stated differently, the tool member 1650 advances through the implant 1100 in a downstream direction and is then withdrawn back through the implant 1100 in an upstream direction to complete a first pass through the implant 1100. In some embodiments, the tool member 1650 can make multiple passes through the implant 1100. In some embodiments, the tool member 1650 performs about 2 to 12 passes to fragment the implant 1100 for removal. In some embodiments, the tool member 1650 traverses through only a segment of the implant 1000 (i.e., less than an entire length of the implant 1000) and the tool member 1650 can traverse the segment of the implant 1000 to partially ablate the implant 1100. The implant 1100 initially placed within the body lumen BL can be any one or more of the biomaterials described herein. The implant 1100 may further be selected to occlude the body lumen BL by interference or frictional fit such that the implant 1100 does not bond or adhere to the inner walls of the body lumen BL. Stated in a different manner, the implant 1100 may be selected such that the tool member 1650 can ablate the implant 1100 and allow portions or fragments 1150 of the implant 1100 to separate from the inner walls without damage to the body lumen BL or perforation of the vessel. The catheter 1510 can be withdrawn and procedure completed while fragments 1150, portions of implant or occlusion are still within the BL. Although the driveshaft 1640 is described as transferring a rotational force to the tool member 1650 to ablate the implant 1100, in some embodiments, the driveshaft and tool member for any of the removal tools described herein can additionally or alternatively be configured to move or oscillate the tool member in a radial and/or axial direction relative to a central axis of the tool member to ablate the implant 1100.

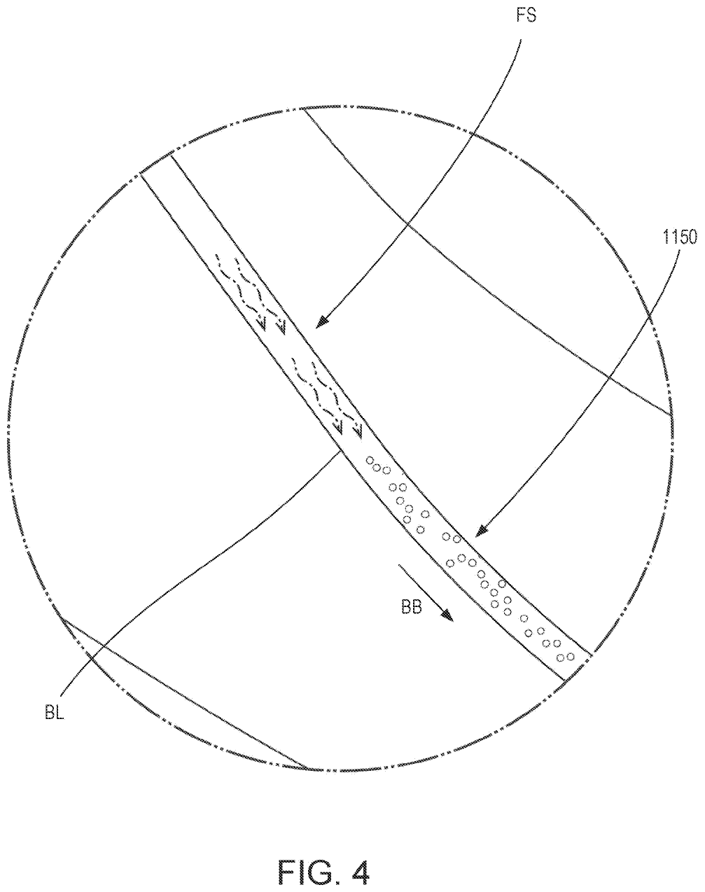

As shown in FIG. 4, once the implant 1100 is ablated into one or more portions or fragments 1150, a flush solution FS may be supplied and conveyed into the body lumen BL in a direction to pass the one or more portions or fragments 1150 and other remnants of the implant 1100 through the body lumen BL. The flush solution FS is introduced upstream of the fragments 1150 such that the fragments are conveyed in a downstream direction (see the arrow BB in FIG. 4) within the body lumen BL. In some embodiments, a flush solution could be administered in an upstream direction and/or in a downstream and upstream direction simultaneously or consecutively administered. For procedures requiring only a single pass of the tool member 1650 through the implant 1100, the flush solution FS can be supplied to the body lumen BL after the single pass has been completed. For procedures requiring multiple passes, the flush solution FS is supplied to the body lumen BL after all of the multiple passes have been completed. In other embodiments, the flush solution FS is supplied to the body lumen BL after each set of two or three passes have been completed and repeated until reversal of the implant 1100 is established. For example, the flush solution FS may be supplied after a first set of two or three passes have been completed. If the flush solution FS passes through the implant location, then the flow passageway through the body lumen BL has been reestablished. If the flush solution FS does not pass through the implant location, or if there is back pressure above a predetermined level when conveying the flush solution FS into the body lumen BL and toward the implant, an additional set of two or three passes followed by a flush can be performed and repeated until the flush solution FS passes through the implant location. In some embodiments, the predetermined level of back pressure is limited by supplying the flush solution FS to the body lumen BL with a force less than or equal to about 44.48 N (10 lbF). In some embodiments, a two sets of two or three passes (i.e., four to six total passes) are performed to sufficiently remove the implant 1100 and to reestablish the flow passageway through the body lumen BL. In some embodiments, three sets of two or three passes (i.e., six to nine total passes) are performed to sufficiently remove the implant 1100 and to reestablish the flow passageway through the body lumen BL. In some embodiments, a total of 2, 3, 4, 5, 6, 7, 8, 9, 10, 11, or 12 passes are performed by the tool member 1650 to ablate the implant 1100. In some embodiments, the implant 1100 may only be partially ablated (e.g., a center core of the implant 1100) via the limited number of passes (i.e., a total of 2, 3, 4, 5, 6, 7, 8, 9, 10, 11, or 12 passes). In some embodiments, only a portion of the ablated implant 1100 is flushed through the body lumen BL with the flushing solution FS, while a remaining portion of the implant 1100 is conveyed through the body lumen aided by natural flow of bodily fluids.

In some embodiments, the body lumen BL is the vas deferens and the flush solution FS is supplied to convey the fragments 1150 and other remnants of the implant 1100 in a downstream direction within the body lumen BL to a urinary tract. In some embodiments, the driveshaft 1640 and the tool member 1650 are withdrawn from the body lumen BL via the guide lumen of the guide member. The flush solution FS is then conveyed to the body lumen via the guide lumen of the guide member. In some embodiments, the guide member, the driveshaft 1640 and the tool member 1650 are all withdrawn from the body lumen BL via the delivery member 1500. The flush solution FS is then supplied to the body lumen BL directly via the delivery member 1500. In some embodiments, a supply tube (not shown) may be inserted into the body lumen BL via the delivery member 1500 and the flush solution FS is conveyed into the body lumen BL through the supply tube. In some embodiments, the flush solution FS is a saline solution and/or water for injection. In some embodiments, the flush solution is a saline solution including a dye. In some embodiments, the dye is a colored dye (e.g., blue, green, orange, red, or yellow) and/or a radiological dye (e.g., iodine-based material, barium-sulfate, gadolinium, and/or saline with air mixture). In some embodiments, the flush solution FS includes an ultrasound-contrast agent such as microbubbles (e.g., bubbles with a diameter of about 3 micron to about 5 micron) and/or nanobubbles (e.g., bubbles with a diameter of less than or equal to about 1 micron). In some embodiments, the flush solution is a phosphate buffered saline. The phosphate buffered saline can include about 0.1 weight % to about 28 weight % sodium chloride or potassium chloride. The phosphate buffered saline can include about 0.01 molar to about 0.3 molar phosphate buffers. In some embodiments, the phosphate buffers can be one or more of Ringer's lactate, citric acid or citrate, tris-hydroxymethyl aminomethane, borate, 2-(N-morpholino)ethanesulfonic acid (MES), acetic acid or acetate.

By supplying the flush solution in a downstream direction, the fragments 1150 are removed from the body lumen BL aided by the normal flow of bodily fluids within the body lumen BL. For example, where the body lumen BL is the vas deferens, the fragments 1150 are flushed downstream and out of the body through the penile urethra. Because the fragments are expelled in this manner, there is no need for a filter, basket or other retrieval tool to be used to capture the fragments 1150 at a downstream location and remove them via the opening through which the catheter 1510 was inserted. Similarly stated, in some embodiments, the methods described herein include ablation of an implant or occlusion to produce fragments without the need for employing a filter or capture device to remove the fragments in an upstream direction. Additionally, in some embodiments, the methods described herein do not employ aspiration to withdraw the fragments 1150 in the upstream direction.

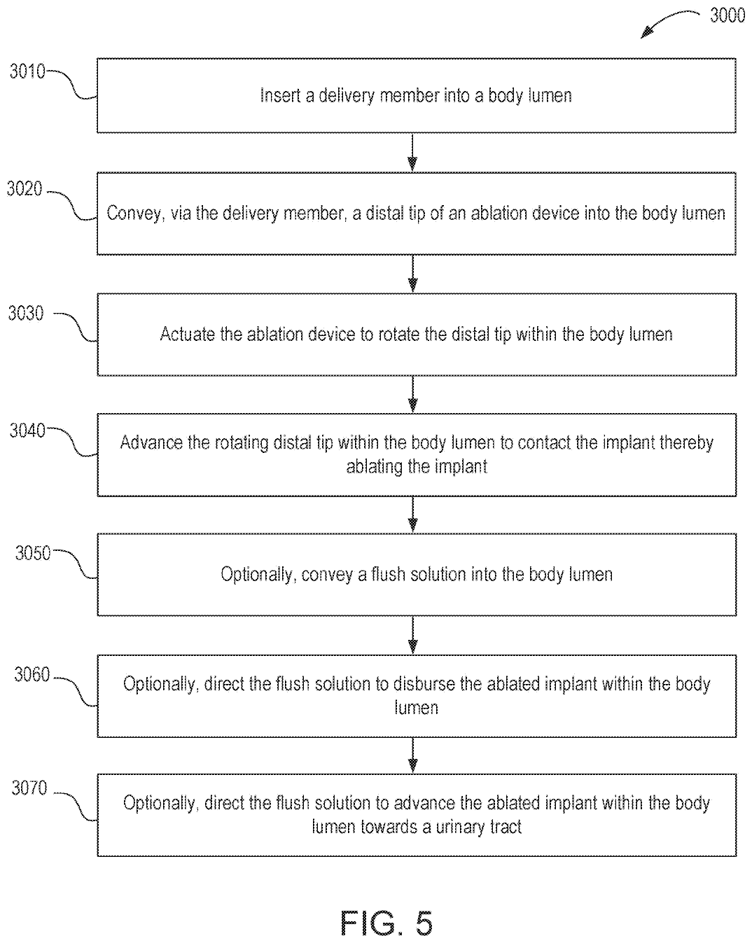

FIG. 5 is a flow chart illustrating a method 3000 of removing an implant from a body lumen. Although the method 3000 is described in connection with the delivery member 1500 and the removal tool 1600 described herein, the method 3000 can be performed with other delivery members and/or removal tools. The method 3000 includes inserting the delivery member 1500 into the body lumen BL, at 3010. In some embodiments, the delivery member 1500 is inserted upstream from the implant 1100 located within the body lumen BL. The method 3000 further includes conveying, via the delivery member 1500, a distal tip portion 1652 of an ablation device 1600 into the body lumen BL, at 3020. The method 3000 includes actuating the ablation device 1600 to rotate the distal tip portion 1652 within the body lumen BL, at 3030. The method 3000 includes advancing the rotating distal tip portion 1652 within the body lumen BL to contact the implant 1100, thereby ablating the implant, at 3040. Optionally, the method 3000 includes conveying a flush solution into the body lumen BL to direct the ablated implant 1100 out of the body lumen BL, at 3050. Optionally, the method includes directing the flush solution to disburse the ablated implant 1100 within the body lumen BL, at 3060. Optionally, the method 3000 includes directing the flush solution to advance the ablated implant 1100 within the body lumen BL towards a urinary tract (e.g., a ureter or a urinary bladder), at 3070.

In some embodiments, the method 3000 further includes conveying the guide member and a driveshaft 1640 of the ablation device 1600 into the body lumen BL via the delivery member 1500. As described above, the guide member includes an outer surface, an inner surface, and a guide lumen defined by the inner surface. The driveshaft 1640 extends through and is rotatably supported by the guide lumen of the guide member. The distal tip portion 1652 of the ablation device 1600 is coupled to a distal end portion of the driveshaft 1640. In some embodiments, the method 3000 further includes actuating a proximal end of the driveshaft 1640 to rotate in a direction about a centerline of the driveshaft 1640. The driveshaft 1640 transfers a rotational force from the proximal end portion to the distal end portion of the driveshaft 1640 to rotate the distal tip portion 1652 of the ablation device 1600.

In some embodiments, the direction of rotation is a first direction about the centerline of the driveshaft 1640 and the rotational force is a first rotational force. The method 3000 further includes actuating the proximal end portion of the driveshaft 1640 to rotate in a second direction about the centerline of the driveshaft 1640, the second direction being opposite the first direction, and the driveshaft 1640 transfers a second rotational force from the proximal end portion to the distal end portion to reverse rotation of the distal tip portion 1652 of the ablation device 1600. In some embodiments, the driveshaft 1640 and/or the guide member are hand guided into the body lumen (BL), through the delivery member 1500, by an operator such as a physician.

In some embodiments, the method 3000 includes withdrawing the driveshaft 1640 and the distal tip portion 1652 of the ablation device 1600 from the body lumen BL through the guide member while the guide member remains inserted into the delivery member 1500. The method 3000 further includes conveying a flush solution into the body lumen BL through the guide lumen of the guide member. In some embodiments, the method includes inserting a supply tube (not shown) into the body lumen BL through the delivery member 1500 and conveying a flush solution into the body lumen BL through the supply tube.

In yet other embodiments, the flush solution can be conveyed via the delivery member 1500. For example, in some embodiments, the hub portion 1520 of the delivery member 1500 can include a luer fitting that can be coupled to (and decoupled from) a syringe. In some embodiments, an implant removal kit can include one (or more) syringes prefilled with flush solution. In use, after the ablation device 1600 is removed from the body lumen BL and the delivery member 1500, the syringe (not shown) can be coupled to the hub portion 1520 of the delivery member 1500, and the flush solution can be conveyed into the body lumen BL directly via the delivery member 1500.

In some embodiments, the method 3000 includes withdrawing the distal tip portion 1652 from the body lumen BL through the delivery member 1500. In some embodiments, the method 3000 further includes inserting a supply tube (not shown) into the body lumen BL through the delivery member 1500. The method 3000 further includes conveying a flush solution into the body lumen BL through the supply tube.

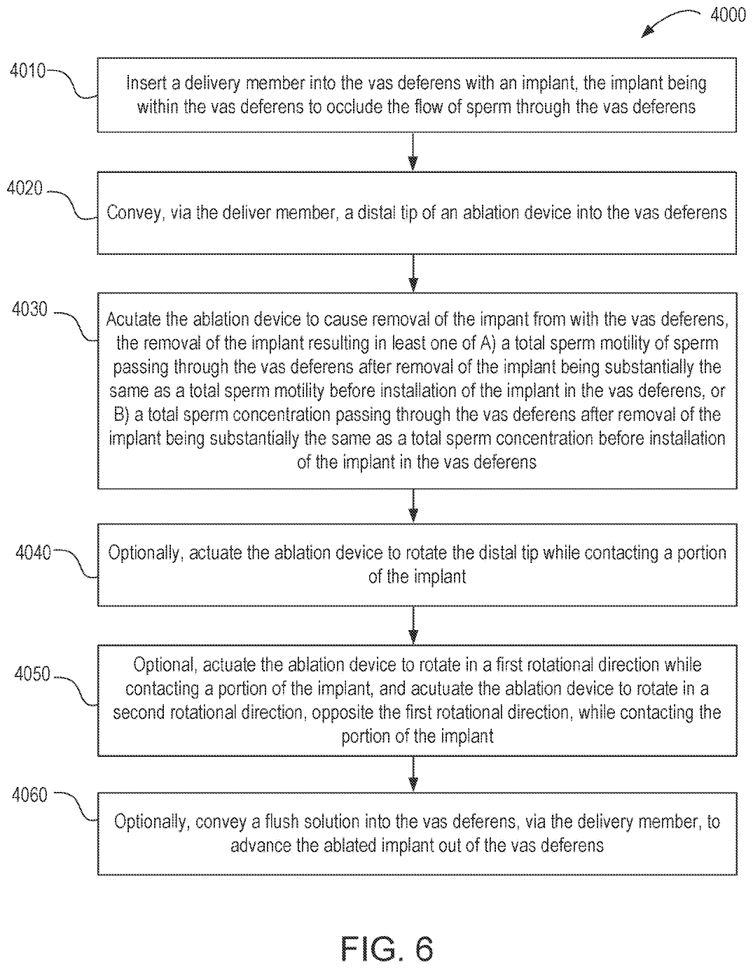

FIG. 6 is a flow chart illustrating a method 4000 of reversing an implant within the vas deferens. The method 4000 can be performed by any delivery members and removal tools, including the delivery members and removal tools described herein. Furthermore, the method 4000 can further include or be substituted with other steps described herein, including steps associated with the method 3000. The method 4000 includes inserting a delivery member into the vas deferens including the implant that has been placed within the vas deferens to occlude the flow of sperm through the vas deferens, at 4010. The method includes conveying a distal tip of an ablation device into the vas deferens via the delivery member, at 4020. The method includes actuating the ablation device to cause disruption, fragmentation, or removal of the implant from within the vas deferens, at 4030. The method further includes actuating the ablation device to cause disruption, fragmentation, or removal of the implant from within the vas deferens. In some embodiments, the disruption, fragmentation, or removal of the implant with the ablation device results in at least one of A) a total sperm motility of sperm passing through the vas deferens after removal of the implant being substantially similar a total sperm motility before placing of the implant in the vas deferens, B) a total sperm concentration passing through the vas deferens after the disruption of the implant being substantially similar to a total sperm concentration before placing of the implant in the vas deferens, C) an ejaculate volume passing through the vas deferens after the disruption of the implant to be substantially similar to the ejaculate volume before placing of the implant in the vas deferens, or D) a forward progression of sperm through the vas deferens after the disruption of the implant being substantially similar to a forward progression of sperm before placing of the implant in the vas deferens. In some embodiments, the disruption, fragmentation, or removal of the implant with the ablation device results in at least one of: A) a total sperm motility of sperm passing through the vas deferens at the implant location after the disruption of the implant being at least 30% to 70% of a total sperm motility of the sperm passing through the vas deferens at a location upstream from the implant location, B) a total sperm concentration passing through the vas deferens at the implant location after the disruption of the implant being at least 30% to 70% of a total sperm concentration passing through the vas deferens at the location upstream from the implant location, or C) an ejaculate volume passing through the vas deferens at the implant location after the disruption of the implant being at least 30% to 70% of an ejaculate volume before passing through the vas deferens at the location upstream from the implant location. In some embodiments, the disruption, fragmentation, or removal of the implant with the ablation device results in at least one of: A) a total sperm motility of sperm passing through the vas deferens at the implant location after the disruption of the implant being at least 30% to 70% of a total sperm motility of the sperm passing through the vas deferens at a location upstream from the implant location, B) a total sperm concentration passing through the vas deferens at the implant location after the disruption of the implant being at least 30% to 70% of a total sperm concentration passing through the vas deferens at the location upstream from the implant location, or C) an ejaculate volume passing through the vas deferens at the implant location after the disruption of the implant being at least 30% to 70% of an ejaculate volume before passing through the vas deferens at the location upstream from the implant location. In some embodiments, the disruption, fragmentation, or removal of the implant with the ablation device results in at least one of A) a total sperm motility of sperm passing through the vas deferens at the implant location after the disruption of the implant being substantially similar to a total sperm motility of the sperm passing through the vas deferens at a location upstream from the implant location, B) a total sperm concentration passing through the vas deferens at the implant location after the disruption of the implant being substantially similar to a total sperm concentration passing through the vas deferens at the location upstream from the implant location, C) an ejaculate volume passing through the vas deferens at the implant location after the disruption of the implant to be substantially similar to an ejaculate volume before passing through the vas deferens at the location upstream from the implant location, or D) a forward progression of sperm through the vas deferens at the implant location after the disruption of the implant being substantially similar to a forward progression of sperm passing through the vas deferens at the location upstream from the implant location. In some embodiments, the disruption, fragmentation, or removal of the implant with the ablation device results in a total sperm motility of sperm passing through the vas deferens at the implant location after the disruption of the implant being at least about 25 micrometers per second. In some embodiments, the disruption, fragmentation, or removal of the implant with the ablation device results in an effective diameter of an inner lumen of the vas deferens at the implant location after the disruption of the implant being substantially similar to the effective diameter of an inner lumen of the vas deferens at a location directly upstream from the implant location. In some embodiments, the disruption, fragmentation, or removal of the implant with the ablation device results in restoration of flow through the vas deferens at the implant location. In some embodiments, the disruption, fragmentation, or removal of the implant with the ablation device enables a flush solution to be conveyed through the implant location of the vas deferens, after the disruption of the implant, with a force of less than or equal to about 44.48 N (10 lbF). In some embodiments, the disruption, fragmentation, or removal of the implant with the ablation device enables at least about 0.1 mL to about 10 mL of flush solution to be conveyed into and through the vas deferens after the disruption of the implant. In some embodiments, “substantially similar” or “substantially the same” in the context of total sperm motility, total sperm concentration, ejaculate volume, or forward progression of sperm, or inner diameter of the vas deferens, includes differences in measurements taken before and after removal or disruption of the implant of up to about 50%, such as a difference of about 5-10%, or a difference of about 10-20%, or a difference of about 20-30%, or a difference of about 30-40%, or a difference of about 40-50%, or any range between any of these endpoints. In embodiments, “substantially similar” includes an increase in any amount from before to after in any one or more of total sperm motility, total sperm concentration, ejaculate volume, or forward progression of sperm. In some embodiments, a test for whether after removal or disruption of an implant the total sperm motility, total sperm concentration, ejaculate volume, or forward progression of sperm is substantially the same as before installation of the implant can be performed in vitro with the implant and removal device, such as in a simulated body lumen, including polyethylene tubing. Such tests can also be performed in other subjects, such as in mice, dogs or rabbits. In some embodiments, “substantially similar” or “substantially the same” in the context of effective diameters of the inner lumen of the vas deferens at the location of the implant and at the location directly upstream of the implant includes a difference of up to about 25%. In some embodiments, the distance from the implant location to the location directly upstream of the implant is about 1 cm. It is noted that depending on the patient, the effective diameter of the inner lumen of the vas deferens at the location of the implant may compress down towards to an original effective diameter (i.e., prior to the presence of the implant) over a period of days, weeks, months, or years after the disruption, fragmentation, or removal of the implant. For some patients, the vas at the implant location may remain dilated beyond the original effective diameter. Optionally, the method 4000 includes actuating the ablation device to rotate the distal tip while contacting a portion of the implant. The rotational contact between the distal tip and the portion of the implant results in fragments being mechanically separated from the implant, at 4040. Optionally, the method 4000 includes actuating the ablation device in a first direction while in contact with the implant, and actuating the ablation device in a second direction, opposite of the first direction, while in contact with the implant, at 4050. For example, the first direction is a first rotational direction and the second direction is a second rotational direction. Optionally, the method 4000 includes conveying a flush solution into the vas deferens, via the delivery member, to advance the ablated implant out of the vas deferens, at 4060.

In some embodiments, the total sperm motility of sperm passing through the vas deferens after removal of the implant is at least about 30% to 70% (e.g., not more than a about 30-50% reduction or difference) the total sperm motility before placing of the implant in the vas deferens. In some embodiments, the total sperm motility of sperm passing through the vas deferens after removal of the implant is at least about 60% to 70% (e.g., not more than about 30-40% reduction or difference) the total sperm motility before placing of the implant in the vas deferens. In some embodiments, the total sperm motility of sperm passing through the vas deferens after removal of the implant is at least about 70% to 80% (e.g., not more than about 20-30% reduction or difference) the total sperm motility before placing of the implant in the vas deferens. In some embodiments, the total sperm concentration passing through the vas deferens after removal of the implant is at least about 85% to 95% (e.g., not more than about 5-15% reduction or difference) the total sperm concentration before placing of the implant in the vas deferens. In some embodiments, the removal of the implant with the ablation device results in a post-implant motility of sperm passing through the vas deferens after removal of the implant that is sufficient to travel through a female reproductive tract and to fertilize an egg. In some embodiments, proteins and organelle (e.g., acrosome) of sperm passing through the vas deferens after the removal of the implant remain unaltered in substantially the same fashion as proteins and organelle of sperm passing through the vas deferens prior to the implant.

In some embodiments, the method 4000 includes advancing the distal tip of the ablation device through at least a central portion of the implant. The method further includes displacing the central portion of the implant to cause at least a portion of an outer perimeter of the implant to contract and collapse towards a center of the vas deferens. The collapse of the implant reduces the effective outer diameter of the implant such that the reduced effective diameter is less than the effective inner diameter of the vas deferens. In some embodiments, the method 4000 further includes advancing the collapsed implant through the vas deferens and toward the urinary tract. In some embodiments, the distal tip of the ablation device includes a diamond coated tip. In some embodiments, the flush solution conveys the fragments of the implant away from the delivery member and in an upstream to downstream direction relative to a direction of flow within the vas deferens towards a urinary tract. In some embodiments, the flush solution includes a saline solution.

In some embodiments, the implant includes an inert, non-biologic material. In some embodiments, the implant is a hydrogel cross-linked in-situ within the vas deferens. In some embodiments, the implant is a cross-linked hydrogel formed from a first component and a second component, the first component includes at least one of a polyvinyl alcohol, alginate or modified alginate, chitosan or modified chitosan, polyethyleneimine, carboxymethyl cellulose, or polyethylene glycol terminated with a biorthogonal functional group. The second component includes at least one of a water or buffer, water or buffer with divalent cations such as calcium, a solution of reduced hyaluronic acid, a solution of polystyrene sulfonate, a solution of gelatin, and/or polyethylene glycol terminated with a biorthogonal functional group.

In some embodiments, the first component includes a multi-arm polyethylene glycol terminated with thiol, and the second component includes a multi-arm polyethylene glycol terminated with a maleimide. In some embodiments, the multi-arm polyethylene glycol terminated with thiol and/or the multi-arm polyethylene glycol terminated with a maleimide have a weight percent ranging from about 1 to 30% in solvent. In some embodiments, the multi-arm polyethylene glycol may be linear, Y-shaped, 3-arm, 4-arm, 6-arm, or 8-arm. In some embodiments, the first component and the second component are formulated to have a pH less than about 5.25.

In some embodiments, the implant can be any one or more of the biomaterials described herein. In some embodiments, the implant is a viscoelastic material. In some embodiments, the delivery member is an angiocatheter. In some embodiments, the body lumen is one of a vas deferens or a fallopian tube. In some embodiments, the body lumen has an inner diameter of 5.0 mm or less and is surrounded by muscle. In some embodiments, the distal tip of the ablation device includes a diamond coated surface. In some embodiments, the flush solution conveyed into the body lumen is a saline solution. In some embodiments, the reversal procedure is performed in 60 minutes or less per vas and the reversal procedure is performed without damage to the inner wall or lining of the vas (epithelial) or perforation of the vessel. In some embodiments, the reversal procedure is performed in 30 minutes or less per vas and the reversal procedure is performed without damage to the inner wall or lining of the vas (epithelial) or perforation of the vessel.

In some embodiments, the method includes reversing the implant in a manner which minimizes damage to the vas deferens, such as an inner lining of the vas deferens (e.g., epithelial lining) or perforation of the vas deferens. The method can result in denuding of columnar nature of the lining, which is capable of regeneration. In some embodiments, the method prevents puncturing, tearing, or shredding of the adjacent muscle layers. For example, in some embodiments, a sensor is coupled to the control unit of the ablation device to detect a load or resistance profile observed the tool such as at the distal tip. If the load or resistance profile is different than a predetermined load or resistance profile representative of the distal tip contacting the implant, the control unit of the ablation device is configured to stop the distal tip from rotating in order to pause or stop the ablation process.

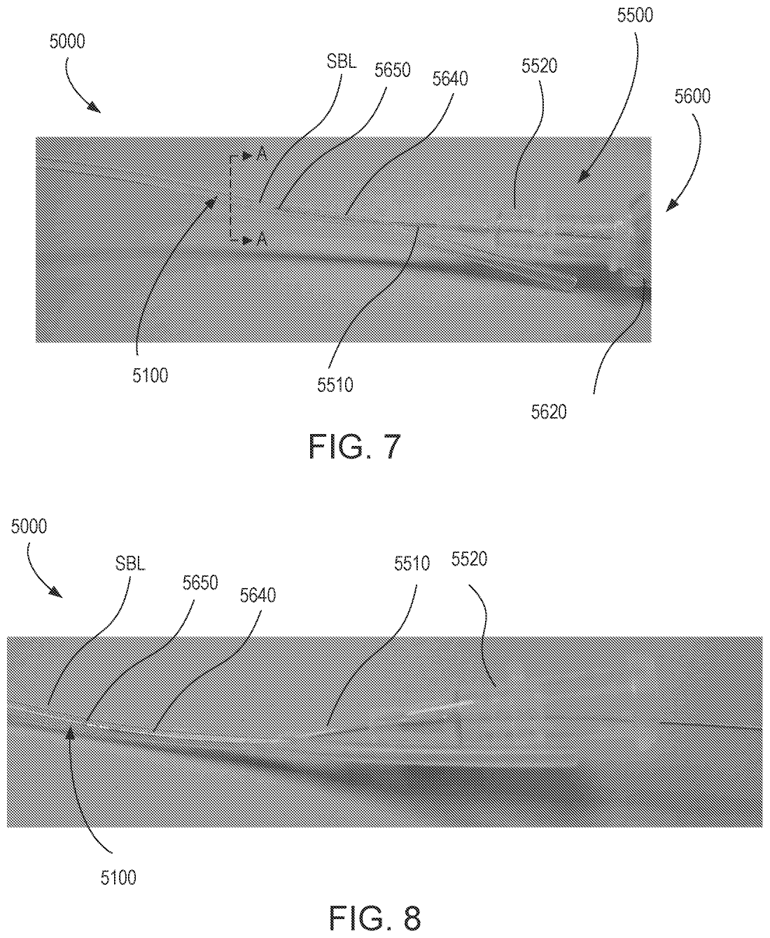

FIGS. 7-10 show views of a test arrangement 5000 including a delivery member 5500, an ablation device 5600, and a simulated body lumen SBL. In particular, the simulated body lumen SBL is a piece of flexible tubing with a 0.8 mm inner diameter and a hydrogel implant 5100 installed to occlude an inner lumen of the simulated body lumen SBL. It is noted that the hydrogel implant 5100 is clear and color-less. The delivery member 5500 is a 24 gauge 0.75-inch over-the-needle catheter including a catheter 5510 and a hub portion 5520. The removal device 5600 includes a motor 5620, a driveshaft 5640, and a tool member 5650. The tool member 5650 includes a diamond coated distal tip surface 5655.

As shown in FIG. 7, the driveshaft 5640 and the tool member 5650 are inserted through the hub portion 5520 and the catheter 5510 of the delivery member 5500 and into the simulated body lumen SBL. The driveshaft 5640 and the tool member 5650 are operable to be inserted into the delivery member 5500 and advanced towards the hydrogel implant 5100.

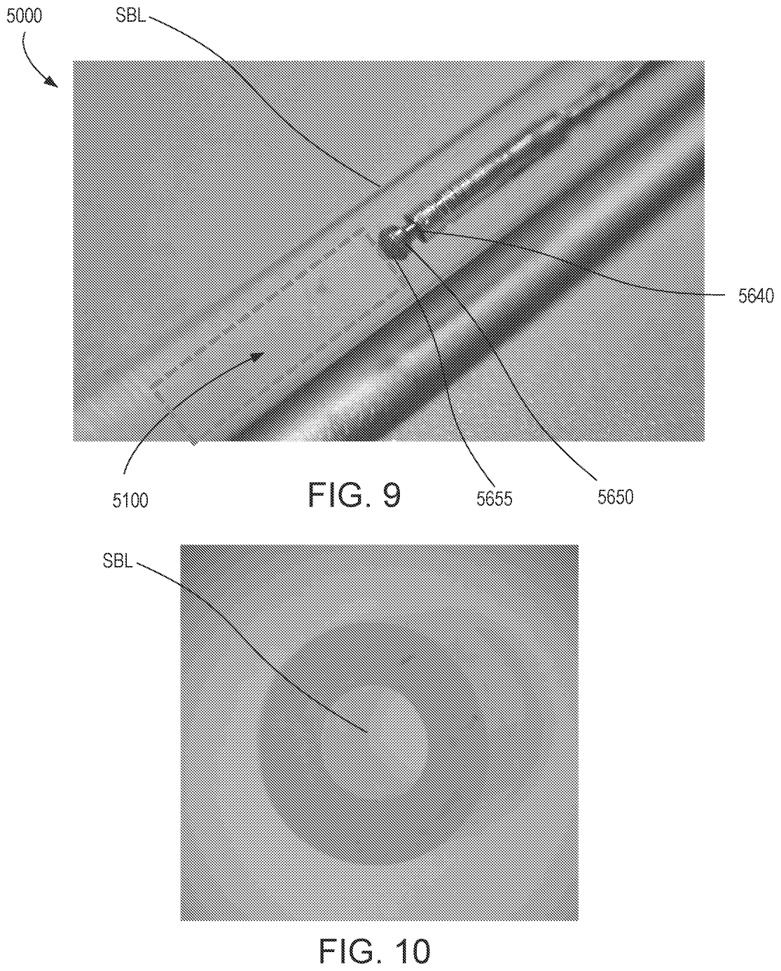

As shown in FIGS. 8 and 9, the tool member 5650 is advanced towards the hydrogel implant 5100 while rotatably driven by the motor 5620 via the driveshaft 5640. As the distal tip surface 5655 of the tool member 5650 contacts the hydrogel implant 5100, fragments of the implant are separated from the original hydrogel implant 5100. As shown in FIG. 10, once the hydrogel implant 5100 has been fragmented and/or ablated by the tool member 5650, the hydrogel implant 5100 can be removed from an inner surface of the simulated body lumen SBL without any damage or alteration to the inner surface of the simulated body lumen SBL. Any of the procedures described herein using any of the tool members described herein can be performed with a simulated body lumen SBL. In some embodiments, the simulated body lumen SBL includes one or more of polyethylene (PE) tubing, polytetrafluoroethylene (PTFE) tubing, polydimethylsiloxane (PDMS) tubing, and/or silicon tubing. For example, the simulated body lumen SBL can be used to calibrate tool member 5650 (or any other tool member described herein) and/or enable a physician or technician to practice removing or reversing an implant outside of a patient.

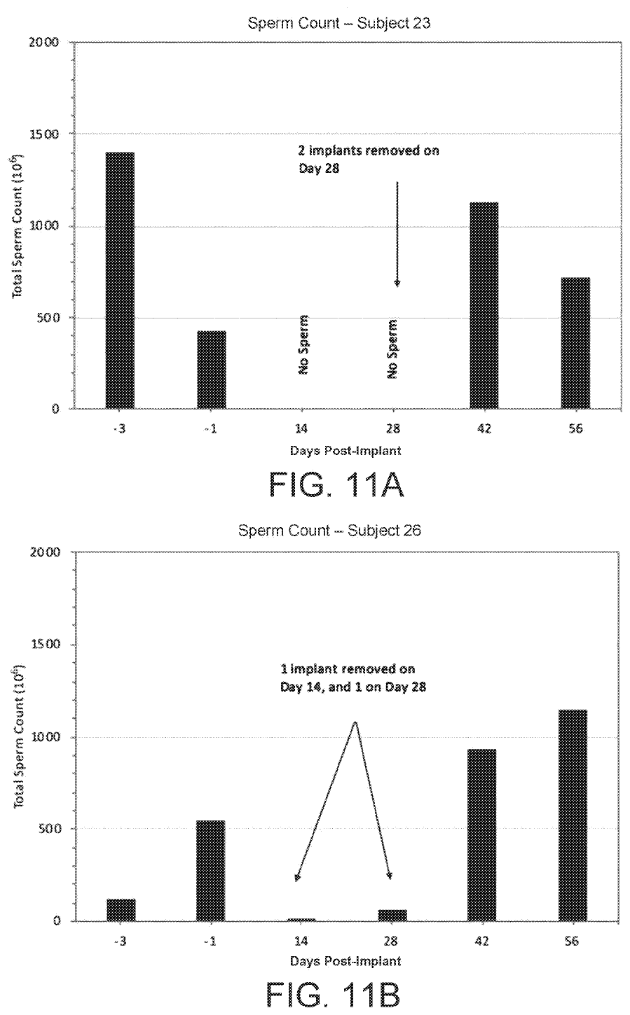

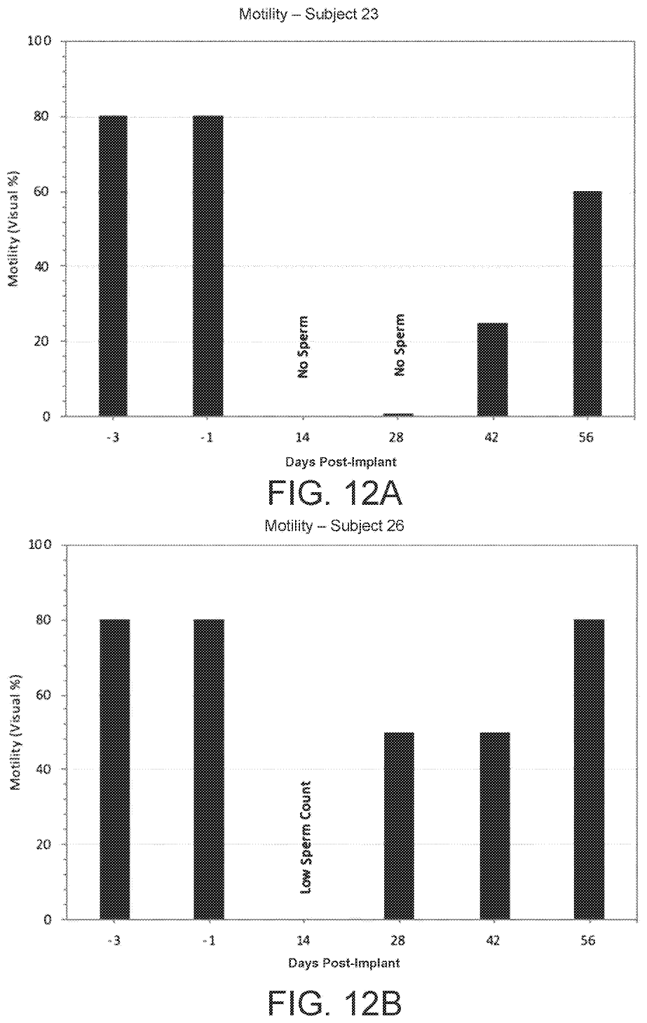

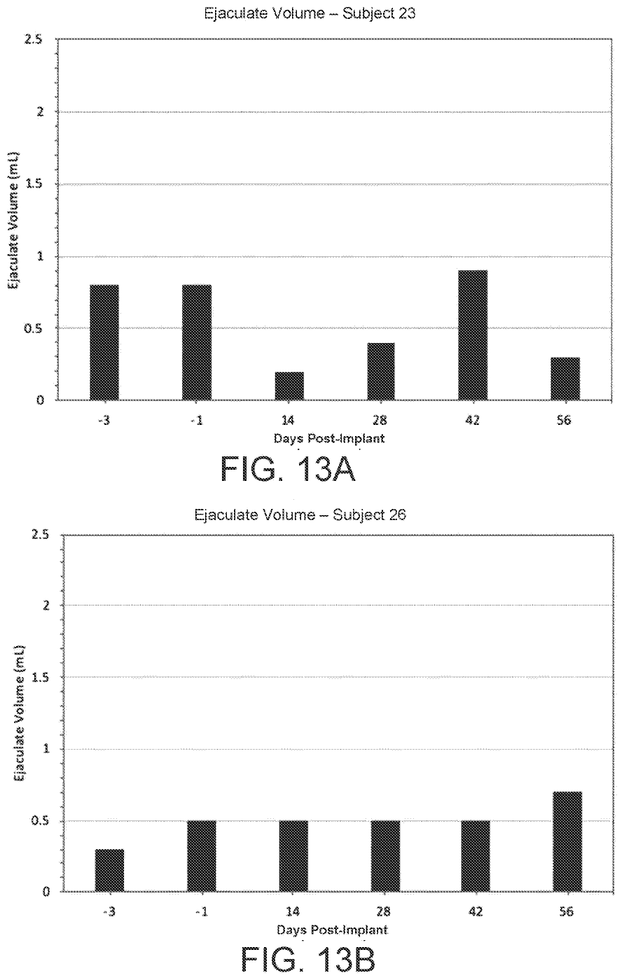

With reference to FIGS. 11A, 11B, 12A, 12B, 13A, and 13B, results are presented for two rabbit test subjects prior to and after in vivo reversal of a non-biologic implant of the types shown and described herein. Although the implants were removed without being ablated using the ablation device 1600 as described herein, the test results are instructive to show the performance of mechanically reversing an implant. Similarly stated, these data show that mechanical methods of removing a non-biologic implant (vs. methods that rely on delivering chemical substances, radiation, or other stimuli to reverse the implant) can be performed in a manner that restores fertility performance to substantially the same as that which was present before placement of the implant. Reversibility was achieved in the two rabbit test subjects after one month of implantation. FIGS. 11A and 11B show sperm count results for the two test subjects prior to the implant being placed in the vas deferens (1 day and 3 days before implant) and also after reversal of the implant using the methods described herein (14 days, 28 days, 42 days and 56 days). After the reversal of the implant, the results indicate that sperm count in the test subjects can be restored to at least within the sperm count range observed prior to the introduction of the implant. FIGS. 12A and 12B show sperm motility results for the two test subjects prior to the implant being placed in the vas deferens (1 day and 3 days before implant) and also after reversal of the implant using the methods described herein (14 days, 28 days, 42 days and 56 days). Within two months after the reversal of the implant, the results indicate that sperm motility is at least about 70% to 90% the total sperm motility observed prior to the introduction of the implant. FIGS. 13A and 13B show ejaculate volume results for the two test subjects prior to the implant being placed in the vas deferens (1 day and 3 days before implant) and also after reversal of the implant using the methods described herein (14 days, 28 days, 42 days and 56 days). Within two months after the reversal of the implant, the results indicate that ejaculate volume can reach ejaculate volume levels observed prior to the introduction of the implant.

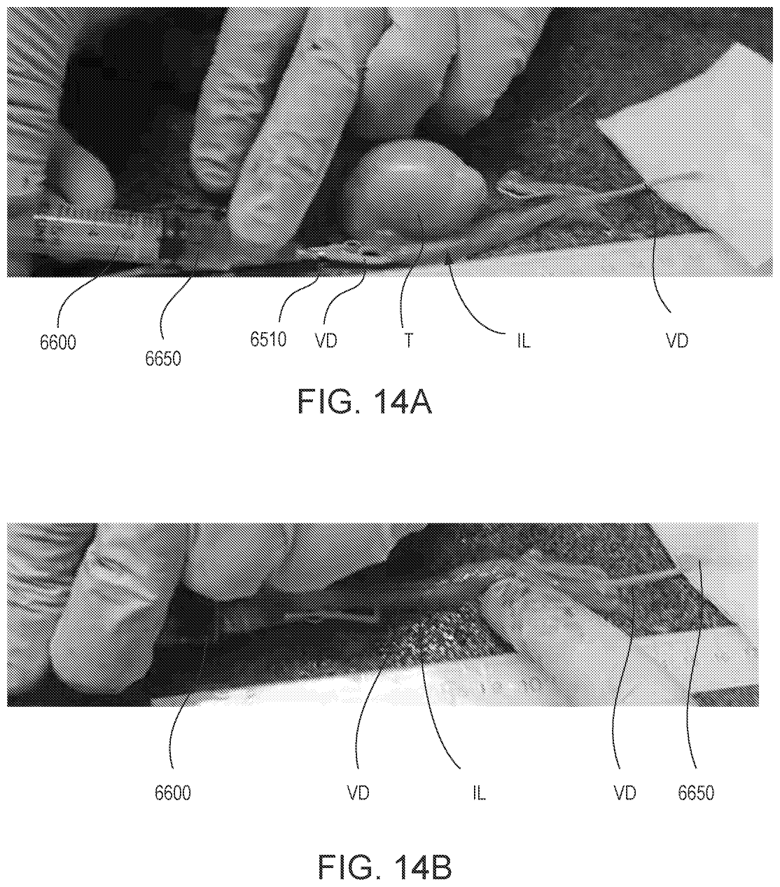

In another test arrangement, a hydrogel implant was formed within a vas deferens VD of a canine subject. The hydrogel implant was subsequently removed from an implant location IL with an ablation device using the methods described herein. As shown in FIG. 14A, tissue including the vas deferens VD and the testis T from the canine subject was excised. A catheter 6510 was inserted into the vas deferens VD behind the implant location IL (i.e., upstream of the implant location IL in the vas deferens VD) where the hydrogel implant was located within the vas deferens VD. A syringe 6600 containing a blue dye solution 6650 was then coupled to the catheter 6510. The blue dye solution 6650 was supplied to the vas deferens VD, via the catheter 6510, behind the implant location IL where the hydrogel implant was located. As shown in FIG. 14B, the blue dye solution 6650 flowed through the vas deferens, traveling through the implant location IL where the hydrogel implant was previously positioned.

In some embodiments, an ablation device includes a distal tip. Use of the ablation device includes removing an implant from a lumen. The distal tip is configured to be conveyed into the lumen via a delivery member that has been inserted into the lumen. The distal tip of the ablation device is configured to be: A) rotated when the ablation device is actuated and B) advanced into the lumen to ablate the implant within the lumen. A flush solution is configured to be conveyed into the lumen. In some embodiments, the flush solution is a saline solution. In some embodiments, the flush solution is a phosphate buffered saline solution.

In some embodiments, the rotating distal tip of the ablation device is configured to be advanced through an entire length of the implant. In some embodiments, the rotating distal tip of the ablation device is configured to be withdrawn through the entire length of the implant. In some embodiments, the rotating distal tip of the ablation device is configured to be advanced through the entire length of the implant a second time after being withdrawn through the entire length of the implant. In some embodiments, the rotating distal tip of the ablation device is configured to be advanced through a central portion of the implant. The rotating distal tip of the ablation device is configured to displace the central portion of the implant to cause at least a portion of an outer perimeter of the implant to contract and collapse towards a center of the lumen.

In some embodiments, the delivery member defines a conduit through which the flush solution is conveyed. The delivery member is configured to direct the flush solution to advance the ablated implant in an upstream to downstream direction relative to a direction of flow within the lumen. In some embodiments, the delivery member defines a conduit and an outlet through which the flush solution is conveyed. The delivery member is configured to direct the flush solution out of the outlet to advance the ablated implant through the lumen and away from the outlet. In some embodiments, the implant includes a viscoelastic material.

In some embodiments, the implant is a hydrogel. In some embodiments, the hydrogel is a cross-linked hydrogel formed from a first component and a second component. In some embodiments, the first component is at least one of a polyvinyl alcohol, alginate or modified alginate, chitosan or modified chitosan, polyethyleneimine, carboxymethyl cellulose, or polyethylene glycol terminated with a biorthogonal functional group, and the second component is at least one of a water or buffer, water or buffer with divalent cations such as calcium, a solution of reduced hyaluronic acid, a solution of polystyrene sulfonate, a solution of gelatin, and/or polyethylene glycol terminated with a biorthogonal functional group.

In some embodiments, the lumen is defined by an inner surface of a tubing material. In some embodiments, the tubing material includes one or more of polyethylene (PE) tubing, polytetrafluoroethylene (PTFE) tubing, polydimethylsiloxane (PDMS) tubing, and/or silicon tubing. In some embodiments, the inner surface of the tubing material has a diameter of about 0.8 mm.

In some embodiments, an ablation device comprises a rotatable distal tip for use in treatment of a blockage within a lumen. The distal tip is configured to be conveyed into the lumen via a delivery member that has been inserted into the lumen. The distal tip of the ablation device is configured to be: A) rotated when the ablation device is actuated and B) advanced into the lumen to ablate the blockage within the lumen. A flush solution is configured to be conveyed into the body lumen to advance the ablated blockage in an upstream to downstream direction relative to a direction of flow within the lumen.

In some embodiments, the blockage is an implant. In some embodiments, the implant includes a hydrogel. In some embodiments, the hydrogel is a cross-linked hydrogel formed from a first component and a second component. In some embodiments, the first component is at least one of a polyvinyl alcohol, alginate or modified alginate, chitosan or modified chitosan, polyethyleneimine, carboxymethyl cellulose, or polyethylene glycol terminated with a biorthogonal functional group, and the second component is at least one of a water or buffer, water or buffer with divalent cations such as calcium, a solution of reduced hyaluronic acid, a solution of polystyrene sulfonate, a solution of gelatin, and/or polyethylene glycol terminated with a biorthogonal functional group.

In some embodiments, the distal tip includes one or more of an abrasive tip, a cutting tip, a milling tip, a grinding tip, a coring tip, or a boring tip. In some embodiments, the distal tip includes one or more a diamond, gold, aluminum, steel, titanium nitride, tungsten carbide, boron carbide, or silica material.

In some embodiments, the lumen is a body lumen. In some embodiments, the body lumen is a vas deferens, and the blockage is an implant within the vas deferens. In some embodiments, the lumen is defined by an inner surface of a tubing material. In some embodiments, the tubing material includes one or more of polyethylene (PE) tubing, polytetrafluoroethylene (PTFE) tubing, polydimethylsiloxane (PDMS) tubing, and/or silicon tubing. In some embodiments, the inner surface of the tubing material has a diameter of about 0.8 mm.

The present disclosure also relates to methods and an apparatus (e.g., removal device, ablation device, etc.) for performing the operations herein. This apparatus may be specially constructed for the intended purposes, or it may comprise a general-purpose computer selectively activated or reconfigured by a computer program stored in the computer. Such a computer program may be stored in a computer readable storage medium, such as, but not limited to, any type of disk including floppy disks, optical disks, CD-ROMs, Universal Serial Bus (USB) flash drives, and magnetic-optical disks, read-only memories (ROMs), random access memories (RAMs), EPROMs, EEPROMs, magnetic or optical cards, or any type of media suitable for storing electronic instructions, each coupled to a computer system bus.

Various general-purpose systems may be used with programs in accordance with the teachings herein, or it may prove convenient to construct a more specialized apparatus to perform the method. The structure for a variety of these systems will appear as set forth in the description below. In addition, the present disclosure is not described with reference to any particular programming language. It will be appreciated that a variety of programming languages may be used to implement the teachings of the disclosure as described herein.

The present disclosure may be provided as a computer program product, or software, that may include a machine-readable medium having stored thereon instructions, which may be used to program a computer system (or other electronic devices) to perform a process according to the present disclosure. A machine-readable medium includes any mechanism for storing information in a form readable by a machine (e.g., a computer). For example, a machine-readable (e.g., computer-readable) medium includes a machine (e.g., a computer) readable storage medium such as a read only memory (“ROM”), random access memory (“RAM”), magnetic disk storage media, optical storage media, flash memory devices, etc.

Some embodiments described herein relate to a computer storage product with a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to: magnetic storage media such as hard disks, floppy disks, and magnetic tape; optical storage media such as Compact Disc/Digital Video Discs (CD/DVDs), Compact Disc-Read Only Memories (CD-ROMs), Universal Serial Bus (USB) flash drives, and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices.

Examples of computer code include, but are not limited to, micro-code or micro-instructions, machine instructions, such as produced by a compiler, code used to produce a web service, and files containing higher-level instructions that are executed by a computer using an interpreter. For example, embodiments may be implemented using imperative programming languages (e.g., C, Fortran, etc.), functional programming languages (Haskell, Erlang, etc.), logical programming languages (e.g., Prolog), object-oriented programming languages (e.g., Java, C++, etc.) or other suitable programming languages and/or development tools. Additional examples of computer code include, but are not limited to, control signals, encrypted code, and compressed code.

A number of embodiments have been described. Nevertheless, it will be understood that various modifications may be made without departing from the spirit and scope of the invention. In addition, the logic flows depicted in the figures do not require the particular order shown, or sequential order, to achieve desirable results. In addition, other steps may be provided, or steps may be eliminated, from the described flows, and other components may be added to, or removed from, the described systems. Accordingly, other embodiments are within the scope of the following claims.

While various embodiments of the invention have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above. Any of the components and sub-components described herein can be included in any of the embodiments unless mutually exclusive. For example, in some embodiments, the methods may be performed successively using a single ablation devices on multiple body lumens, such as a first vas deferens and a second vas deferens of a patient.

In some embodiments, biomaterial forming the implant in the body lumen is formed from one or more precursors. For example, two macromer solutions are injected that cross-link with each other to form a hydrogel material. The delivery apparatus injects solutions into the body, such that the solutions form a hydrogel in situ. In some embodiments, the delivery apparatus is used to inject the formed biomaterial into the body, e.g. cross-linked hydrogel. The hydrogel may continue to gel and/or cross-link in situ once injected or can be completely gelled or cross-linked by the time it exits the delivery apparatus. In this regard, the delivery apparatus facilitates the merging or mixing of the two or more different solutions into a single stream.

In some embodiments, the biomaterial forming the implant in the body lumen includes one or more of natural or synthetic monomers, polymers or copolymers, biocompatible monomers, polymers or copolymers, such as polystyrene, neoprene, polyetherether ketone (PEEK), carbon reinforced PEEK, polyphenylene, polyetherketoneketone (PEKK), polyaryletherketone (PAEK), polyphenylsulphone, polysulphone, polyurethane, polyethylene, low-density polyethylene (LDPE), linear low-density polyethylene (LLDPE), high-density polyethylene (HDPE), polypropylene, polyetherketoneetherketoneketone (PEKEKK), nylon, fluoropolymers such as polytetrafluoroethylene (PTFE or TEFLON®), TEFLON® TFE (tetrafluoroethylene), polyethylene terephthalate (PET or PETE), TEFLON® FEP (fluorinated ethylene propylene), TEFLON® PFA (perfluoroalkoxy alkane), and/or polymethylpentene (PMP) styrene maleic anhydride, styrene maleic acid (SMA), polyurethane, silicone, polymethyl methacrylate, polyacrylonitrile, poly(carbonate-urethane), poly(vinylacetate), nitrocellulose, cellulose acetate, urethane, urethane/carbonate, polylactic acid, polyacrylamide (PAAM), poly(N-isopropylacrylamine) (PNIPAM), poly(vinylmethylether), poly(ethylene oxide), poly(ethyl (hydroxyethyl) cellulose), poly(2-ethyl oxazoline), polylactide (PLA), polyglycolide (PGA), poly(lactide-co-glycolide) PLGA, poly(e-caprolactone), polydiaoxanone, polyanhydride, trimethylene carbonate, poly(β-hydroxybutyrate), poly(g-ethyl glutamate), poly(DTH-iminocarbonate), poly(bisphenol A-iminocarbonate), poly(orthoester) (POE), polycyanoacrylate (PCA), polyphosphazene, polyethyleneoxide (PEO), polyethylene glycol (PEG) or any of its derivatives, polyacrylacid (PAA), polyacrylonitrile (PAN), polyvinylacrylate (PVA), polyvinylpyrrolidone (PVP), polyglycolic lactic acid (PGLA), poly(2-hydroxypropyl methacrylamide) (pHPMAm), poly(vinyl alcohol) (PVOH), PEG diacrylate (PEGDA), poly(hydroxyethyl methacrylate) (pHEMA), N-isopropylacrylamide (NIPA), polyoxazoline (POx), poly(vinyl alcohol) poly(acrylic acid) (PVOH-PAA), collagen, silk, fibrin, gelatin, hyaluron, cellulose, chitin, dextran, casein, albumin, ovalbumin, heparin sulfate, starch, agar, heparin, alginate, fibronectin, keratin, pectin, elastin, ethylene vinyl acetate, ethylene vinyl alcohol (EVOH), polyethylene oxide, PLLA or PLA (poly(L-lactide) or poly(L-lactic acid)), poly(D,L-lactic acid), poly(D,L-lactide), polydimethylsiloxane or dimethicone (PDMS), poly(isopropyl acrylate) (PIPA), polyethylene vinyl acetate (PEVA), PEG styrene, polytetrafluoroethylene RFE such as TEFLON® RFE or KRYTOX® RFE, fluorinated polyethylene (FLPE or NALGENE®), methyl palmitate, temperature responsive polymers such as poly(N-isopropylacrylamide) (NIPA), polycarbonate, polyethersulfone, polycaprolactone, polymethyl methacrylate, polyisobutylene, nitrocellulose, medical grade silicone, cellulose acetate, cellulose acetate butyrate, polyacrylonitrile, poly(lactide-co-caprolactone (PLCL), and/or chitosan.

In some embodiments, the dissolving solution for the polymer component(s) may be aqueous buffers (pH range 1-14): phosphate, citrate, acetate, histidine, lactate, tromethamine, gluconate, aspartate, glutamate, tartrate, succinate, malic acid, fumaric acid, alpha-ketoglutaric, and/or carbonate. Non-aqueous solvents include: dimethyl isosorbide, glycofurol 75, PEG 200, diglyme, tetrahydrofurfuryl alcohol, ethanol, acetone, solketal, glycerol formal, dimethyl sulfoxide, propylene glycol, ethyl lactate, N-methyl-2-pyrrolidone, dimethylacetamide, methanol, isopropanol, 1,4-butanediol, ethyl acetate, toluene, acetonitrile. In some embodiments, when the polymer component is dissolved, the viscosity of the solution(s) that make up the biomaterial may range from about 0.1 to about 250,000 cP, such as about 0.5 to about 200,000 cP, about 1 to about 150,000 cP, about 5 to about 100,000 cP, about 10 to about 75,000 cP, about 20 to about 50,000 cP, about 50 to about 25,000 cP, about 100 to about 10,000 cP, about 500 to about 7,500 cP, or about 1,000 to about 5,000 cP, or any viscosity in between. The density of the solution may range from about 0.1 to about 20,000 kg/m³, such as about 1 to about 15,000 kg/m³, about 5 to about 12,500 kg/m³,about 10 to about 10,000 kg/m³, about 100 to about 5,000 kg/m³, about 500 to about 2,5000 kg/m³, or about 1,000 to about 1,500 kg/m³, or any density in between. The temperature during extrusion may range from about 2 to about 45° C., such as about 5 to about 40° C., about 10 to about 38 ° C., about 15 to about 37° C., about 20 to about 36° C., about 25 to about 35° C., about 30 to about 34° C., or about 31 to about 33° C., or any temperature in between. The pH of the solution(s) may range from 1-14. The ionic strength of the solution(s) may range from about 1 nM to about 70 M, such as about 5 nM to about 60 M, about 10 nM to about 50 M, about 20 nM to about 25 M, about 50 nM to about 15 M, about 75 nM to about 10 M, 100 nM to about 5 M, or about 500 nM to about 2.5 M, or any molarity in between.

In some embodiments, if two components are injected to form the biomaterial, then the ratio of the components may be varied such as 1:1, 2:1, 1:2, 3:1, 1:3, 4:1, 1:4, and up to 10:1 or 1:10. The gelation time of the biomaterial may range from about 0.001 seconds to about 60 minutes, such as about 1 second to about 45 minutes, about 5 seconds to about 30 minutes, about 10 seconds to about 15 minutes, about 20 seconds to about 10 minutes, about 30 seconds to about 8 minutes, about 45 seconds to about 5 minutes, about 1 minute to about 3 minutes, or about 1.5 minutes to about 2.5 minutes, or any range in between. The length of the formed biomaterial may range from about 0.1 to about 60 cm, such as about 0.2 to about 50 cm, about 0.3 to about 40 cm, about 0.4 to about 30 cm, about 0.5 to about 20 cm, about 0.6 to about 15 cm, about 0.8 to about 10 cm, about 0.9 to about 5 cm, about 1.2 to about 4 cm, about 1.4 to about 3 cm, about 1.6 to about 2.5 cm, or about 1.8 to about 2.2 cm, or any range in between. The volume of the formed biomaterial may range from about 0.001 to about 100 mL, such as about 0.005 to about 90 mL, about 0.01 to about 80 mL, about 0.05 to about 70 mL, about 0.1 to about 60 mL, about 0.2 mL to about 50 mL, about 0.25 to about 40 mL, about 0.4 to about 30 mL, about 0.5 to about 20 mL, about 0.7 to about 10 mL, about 0.9 to about 5 mL, about 1.1 to about 4 mL, about 1.4 to about 3 mL, or about 2 mL to about 2.5 mL, or any range in between.

In some embodiments, the biomaterial forming the implant swells within the implantation space to lock or secure its placement. For example, a biomaterial in the form of a hydrogel may swell from about 1.5× to about 10× its initial volume, such as about 2× to about 8×, about 2.5× to about 7×, about 3× to about 6×, or about 4× to about 5×. In some embodiments, the extruded biomaterial conforms to the space it is injected into. In some embodiments, the swelling of the biomaterial does not change volume within the implantation space, or shrinks to conform to a volume of the implantation space. In some embodiments, the implant is injected or delivered as a pre-formed biomaterial (does not cross-link, form, or gel in situ). Once injected, the biomaterial may or may not react with the implantation space. If a reaction does occur, it may be covalent or non-covalent. In some embodiments, the biomaterial adhesively interacts within the implantation space.

Although the control unit 1610 is described as receiving inputs from an operator, the control unit 1610 may be operably connected to one or more sensor, network modules and/or other processing modules to vary the output of the motor 1620 based on sensed conditions, remote control, and/or programmed control.

Although various embodiments have been described as having particular features and/or combinations of components, other embodiments are possible having a combination of any features and/or components from any of embodiments where appropriate.

1.(canceled)

2. The method of claim 7, wherein the advancing of the distal tip includes moving the distal tip through an entire length of the implant.

3-4. (canceled)

5. The method of claim 7, further comprising:
directing the flush solution to advance the ablated implant in an upstream to downstream direction relative to a direction of flow within the body lumen.

6. The method of claim 5, wherein the directing of the flush solution includes advancing the ablated implant to a portion of a urinary tract.

7. A method, comprising:
inserting a delivery member into a body lumen;
conveying, via the delivery member, a distal tip of an ablation device into the body lumen;
actuating the ablation device to rotate the distal tip within the body lumen;
advancing the distal tip, while the distal tip is rotating, within the body lumen to contact the implant thereby ablating the implant;
conveying a flush solution into the body lumen;
withdrawing, via the delivery member, the distal tip from the body lumen;
inserting, via the delivery member, a supply tube into the body lumen; and
conveying, via the supply tube, the flush solution into the body lumen.

8-9. (canceled)

10. The method of claim 7, wherein the body lumen is less than 5 mm in diameter.

11. The method of claim 7, wherein the flush solution includes a saline solution.

12. The method of claim 7, wherein the implant is a hydrogel.

13-20. (canceled)

21. A method, comprising:
inserting a delivery member into a vas deferens, an implant being disposed within the vas deferens at an implant location to occlude a flow of sperm through the vas deferens;
conveying, via the delivery member, a distal tip of an ablation device into the vas deferens;
actuating the ablation device to cause disruption of the implant from within the vas deferens; and
wherein disruption of the implant by the ablation device results in a total sperm motility of sperm passing through the vas deferens at the implant location after the disruption of the implant being at least about 25 micrometers per second.

22. The method of claim 21, wherein disruption of the implant by the ablation device results in an effective diameter of an inner lumen of the vas deferens at the implant location after the disruption of the implant being substantially similar to the effective diameter of an inner lumen of the vas deferens at a location directly upstream from the implant location.

23. The method of claim 21, wherein the disruption of the implant with the ablation device enables a flush solution to be conveyed through the implant location of the vas deferens, after the disruption of the implant, with a force of less than or equal to about 44.48 N (10 lbF).

24. The method of claim 21, wherein the disruption of the implant with the ablation device enables at least about 0.1 mL to about 10 mL of a flush solution to be conveyed into and through the implant location of the vas deferens after the disruption of the implant.

25. The method of claim 21, wherein the actuating of the ablation device includes rotating the distal tip while contacting a portion of the implant, the rotational contact between the distal tip and the portion of the implant causing fragments from the portion of the implant to be mechanically separated from the implant.

26. The method of claim 21, further comprising:
conveying, via the delivery member, a flush solution into the vas deferens to advance one or more of the fragments out of the vas deferens.

27. The method of claim 21, wherein the flush solution conveys one or more of the fragments away from the delivery member in an upstream to downstream direction relative to a direction of flow within the vas deferens and towards a urinary tract.

28-29. (canceled)

30. A method, comprising:
inserting a delivery member into a vas deferens, an implant being disposed within the vas deferens at an implant location to occlude a flow of sperm through the vas deferens;
conveying, via the delivery member, a distal tip of an ablation device into the vas deferens;
actuating the ablation device to cause disruption of the implant from within the vas deferens;
advancing the distal tip through at least a central portion of the implant; and
displacing the central portion of the implant to cause at least a portion of an outer perimeter of the implant to contract and collapse towards a center of the vas deferens.

31. The method of claim 30, wherein collapsing the outer perimeter of the implant causes the outer perimeter to separate from the vas deferens without damage to the vas deferens.

32. The method of claim 30, further comprising:
advancing the implant with the collapsed outer perimeter through the vas deferens and towards a urinary tract.

33. The method of claim 30, wherein the actuating of the ablation device includes moving the distal tip in a first direction while in contact with the implant, and moving the distal tip in a second direction while in contact with the implant.

34-61. (canceled)
